(19) United States
(12) Patent Application Publication
(10) Pub. No.: US 2022/0267714 A1
(43) Pub. Date: Aug. 25, 2022

(54) METHOD OF OBTAINING MITOCHONDRIA FROM CELLS AND OBTAINED MITOCHONDRIA

(71) Applicant: LUCA SCIENCE INC., Tokyo (JP)

(72) Inventors: Yoshihiro OHTA, Tokyo (JP); Arima OKUTANI, Tokyo (JP); Momoka TAKAHASHI, Tokyo (JP); Masashi SUGANUMA, Tokyo (JP); Yoshie KAWASE, Tokyo (JP)

(21) Appl. No.: 17/628,834

(22) PCT Filed: Jul. 22, 2020
(86) PCT No.: PCT/JP2020/029597

§ 371 Date: Jan. 20, 2022

(30) Foreign Application Priority Data
Jul. 24, 2019 (JP) 2019-136283

(51) Int. Cl. C12N 1/06; C12N 5/071

(52) U.S. Cl. CPC C12N 1/06; C12N 5/0602; C12N 2509/00

(57) ABSTRACT

The present disclosure relates to methods of obtaining mitochondria from cells, mitochondria obtained by such methods, and uses of mitochondria obtained by such methods.

### CROSS REFERENCE TO RELATED APPLICATIONS

This application claims the benefit of priority to Japan application number 2019-136283, filed on Jul. 24, 2019, the entire contents of which are hereby incorporated by reference.

### BACKGROUND

Mitochondria are a type of organelle that plays three key roles: 1) metabolism such as ATP synthesis, 2) intracellular signaling such as Ca2+ and reactive oxygen species, and 3) control of cell death such as apoptosis and necrosis. In this sense, mitochondria are strongly associated with disease and have been studied by many researchers from a health perspective.

For mitochondrial function, the folded inner membrane and the surrounding outer membrane, and the electron transport system located in the inner membrane play a crucial role. The inner membrane forms a highly folded structure called cristae, which is believed to hold the supercomplex of electron transport system in the cristae membrane and to keep the proton concentration high by trapping the pumped protons in the cristae space. The electrochemical proton gradient formed by the electron transport system enables the transport of anions as well as ATP synthesis and cation transport.

Decreased mitochondrial function can cause a variety of diseases. There are currently no methods known in the art for isolating mitochondria from cells in a manner that retains mitochondrial function and structural integrity. This disclosure addresses this and other needs.

### BRIEF SUMMARY

The present disclosure provides a population of isolated or obtained or processed mitochondria, wherein the mitochondria in the population exhibit superior functional capability. For example, in an aspect, the present disclosure provides a population of isolated mitochondria wherein at least about 60%, at least about 65%, at least about 70%, at least about 75%, at least about 80%, at least about 85%, at least about 90%, or at least about 95% of the mitochondria in the population have intact inner and outer membranes; and/or at least about 60%, at least about 65%, at least about 70%, at least about 75%, at least about 80%, at least about 85%, at least about 90%, or at least about 95% of the mitochondria in the population are polarized as measured by a fluorescence indicator. In embodiments, the fluorescence indicator is selected from the group consisting of positively charged dyes such as JC-1, tetramethylrhodamine methyl ester (TMRM), and tetramethylrhodamine ethyl ester (TMRE).

In embodiments, the present disclosure provides a population of isolated mitochondria, wherein at least about 60%, at least about 65%, at least about 70%, at least about 75%, at least about 80%, at least about 85%, at least about 90%, or at least about 95% of the mitochondria in the population maintain functional capability (e.g., are polarized) in an extracellular environment. In embodiments, the functional capability in an extracellular-environment is measured by a fluorescence indicator of membrane potential. In embodiments, the fluorescence indicator is selected from the group consisting of positively charged dyes such as JC-1, TMRM, and TMRE. In embodiments, the extracellular environment may comprise a total calcium concentration of about 4 mg/dL to about 12 mg/dL, or about 1 mmol/L (1000 μM) to about 3 mmol/L (3000 μM). For example, in embodiments, the extracellular environment comprises a concentration of total calcium of about 8 mg/dL to about 12 mg/dL, or about 2 mmol/L (2000 μM) to about 3 mmol/L (3000 μM). In embodiments, the extracellular environment comprises a concentration of free or active calcium of about 4 mg/dL to about 6 mg/dL, or about 1 mmol/L (1000 μM) to about 1.5 mmol/L (1500 μM). In embodiments, the population of mitochondria maintain functional capability in an environment having a higher calcium concentration compared to the calcium environment in a cell.

In embodiments, provided herein is a population of isolated mitochondria wherein at least about 60%, at least about 65%, at least about 70%, at least about 75%, at least about 80%, at least about 85%, at least about 90%, or at least about 95% of the mitochondria in the population are not undergoing dynamin-related protein 1 (drp1)—dependent division. In embodiments, provided herein is a population of isolated mitochondria having inner and outer membranes, wherein the inner membranes of the mitochondria comprise densely folded cristae.

In embodiments, provided herein is a population of isolated mitochondria wherein at least about 60%, at least about 65%, at least about 70%, at least about 75%, at least about 80%, at least about 85%, at least about 90%, or at least about 95% of the mitochondria in the population have a substantially non-filamentous, non-branched structure or shape. For example, in embodiments, the mitochondria provided herein appear as round, dot-like, globular, irregularly shaped, and/or slightly elongated, or any mixture thereof, when viewed under a microscope. In embodiments, at least about 60%, at least about 65%, at least about 70%, at least about 75%, at least about 80%, at least about 85%, at least about 90%, or at least about 95% of the mitochondria in the population have a longer diameter to shorter diameter ratio of no more than 4:1, no more than 3.5:1, or no more than 3:1. In embodiments, at least about 60%, at least about 65%, at least about 70%, at least about 75%, at least about 80%, at least about 85%, at least about 90%, or at least about 95% of the isolated mitochondria in the population of mitochondria provided herein have a length shorter than the double or triple of the hydrodynamic diameter of the mitochondrion. In this manner, the isolated mitochondria provided herein have a markedly different shape (non-filamentous) when compared to the shape of most mitochondria (filamentous) that are within cells. Thus, in embodiments, the population of mitochondria provided herein has a shape that is distinct from mitochondria that exist in a cell and have not been isolated, in that at least about 60%, at least about 65%, at least about 70%, at least about 75%, at least about 80%, at least about 85%, at least about 90%, or at least about 95% of the mitochondria in the population are non-filamentous in shape. In embodiments, the population of isolated mitochondria provided herein exhibit decreased association with mitochondria-associated membrane (MAM). In embodiments, the association with MAM is measured by expression of glucose regulated protein 75 (GRP75). In embodiments, the population of isolated mitochondria provided herein exhibit about 60%, at least about 65%, at least about 70%, about 60%, about 50%, about 40%, about 30%, or less association with MAM when compared to mitochondria in a cell, and/or mitochondria that have been obtained by a conventional method of isolation such as one involving homogenization and/or high levels of detergent, as further described herein. In embodiments, the population of isolated mitochondria provided herein exhibit a decrease in association with MAM, wherein the decrease is at least about 30%, at least about 40%, at least about 50%, at least about 60%, at least about 70%, or more relative to the association with MAM of mitochondria in a cell or of mitochondria isolated by a conventional method of isolation.

In embodiments, the population of isolated mitochondria provided herein are between about 500 nm and about 3500 nm in size. In embodiments, at least about 60%, at least about 65%, at least about 70%, at least about 75%, at least about 80%, at least about 85%, at least about 90%, at least about 95%, or at least about 99% of the mitochondria in the population are between about 500 nm and about 3500 nm in size. In embodiments, the average size of the mitochondria in the population is about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, or about 3500 nm. In embodiments, the polydispersity index (PDI) of the population of isolated mitochondria is about 0.2 to about 0.8. In embodiments, the PDI of the population of isolated mitochondria is about 0.2 to about 0.5. In embodiments, the PDI of the population of isolated mitochondria is about 0.25 to about 0.35. In embodiments, the zeta potential of the population of mitochondria is about −15 mV to about −40 mV. In embodiments, the zeta potential of the population of mitochondria is about −20 mV, about −25 mV, about −30 mV, about −35 mV, or about −40 mV.

In embodiments, the population of isolated mitochondria provided herein are capable of being incorporated into cells and/or co-localization with endogenous mitochondria in cells, when the population of isolated mitochondria is contacted with a population of cells. For example, in embodiments, the present disclosure provides methods for obtaining mitochondria from cells, and subsequently contacting a population of cells (e.g., ex vivo or in vivo cells) with the population of isolated mitochondria. In such embodiments, the mitochondria provided herein, which are isolated via the iMIT method described herein, are capable of co-localizing with the endogenous mitochondria present in the cells. In embodiments, the mitochondria provided herein are further capable of fusing with the mitochondria present in the cells that they have contacted. In embodiments, a substantial fraction of the population of isolated mitochondria are capable of co-localization and/or fusion with endogenous mitochondria in cells. For example, in embodiments, at least about 30%, at least about 40%, at least about 50%, at least about 60%, at least about 70%, at least about 80%, at least about 90%, or at least about 95% of the mitochondria in the population are capable of co-localization and/or fusion with endogenous mitochondria in cells. Thus, the mitochondria provided herein are markedly different from mitochondria isolated via conventional methods in that they are capable of co-localization and/or fusion with endogenous mitochondria in cells.

In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about 4° C. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about 4° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −20° C. or colder. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −20° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −80° C. or colder. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −80° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage in liquid nitrogen. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage in liquid nitrogen. In embodiments, the storage is for at least about 2 hours, at least about 6 hours, at least about 12 hours, at least about 24 hours, at least about 48 hours, at least about 1 week, at least about 2 weeks, at least about 3 weeks, at least about 1 month, at least about 2 months, at least about 3 months, or longer. Thus, in embodiments, the isolated mitochondria provided herein are markedly different from mitochondria isolated via conventional methods at least in that they maintain functional capacity when freshly isolated and even after storage.

In embodiments, the isolated mitochondria provided herein are stable and/or polarized and/or maintain membrane potential and/or maintain an intact inner and outer membrane and/or-maintain the capacity to function after exposure to an extracellular environment (e.g., after exposure to a total calcium concentration of about 4 mg/dL to about 12 mg/dL), after the population of mitochondria have been frozen for storage and then thawed. In embodiments, after being frozen and then thawed, the maintenance rate of the membrane potential is about 90% relative to the membrane potential of the mitochondria prior to freezing. For example, in embodiments, the polarization ratio of a population of mitochondria that has been frozen and thawed is about 90% of the polarization ratio of that population prior to freezing. In embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after being frozen for storage and then thawed, for example, after being frozen for storage and then thawed one, two, three, or more times. Thus, in embodiments, the isolated mitochondria provided herein are markedly different from mitochondria isolated via conventional methods at least in that they maintain functional capacity when even after being frozen for storage and then thawed.

In embodiments, the population of isolated mitochondria provided herein are capable of being incorporated into cells and/or co-localization with and/or fusion with endogenous mitochondria in cells after storage of the mitochondria at any temperature provided herein (e.g., 4° C.±3° C., −20° C.±3° C., −80° C.±3° C., or in liquid nitrogen). For example, in embodiments, at least about 60%, at least about 65%, at least about 70%, at least about 75%, at least about 80%, at least about 85%, at least about 90%, or at least about 95% of the mitochondria in the population capable of being incorporated into cells and/or co-localization with and/or fusion with endogenous mitochondria in cells after the mitochondria have been stored and/or undergone one or more freeze-thaw cycle. In embodiments, the method of storing and thawing the population of isolated mitochondria provided herein comprises storing the population at about −20° C.±3° C., about −80° C.±3° C., or colder (e.g., in liquid nitrogen), and then thawing the mitochondria at about 20° C.±3° C. or colder, wherein the mitochondria are thawed within about 5 minutes, about 4 minutes, about 3 minutes, about 2 minutes, or about 1 minute. In particular embodiments, the population of mitochondria is thawed within about 1 minute. Thus, in embodiments, the mitochondria provided herein are markedly different from mitochondria isolated via conventional methods at least in that they are capable of being incorporated into cells and/or co-localization with and/or fusion with endogenous mitochondria in cells, whereas mitochondria isolated by conventional methods are incapable of or exhibit vastly reduced ability to being incorporated into cells and/or co-localize with and/or fuse with endogenous mitochondria in cells. In embodiments, the co-localized isolated mitochondria can form a filamentous structure, a network structure, and/or a mesh-like structure.

In embodiments, the present disclosure provides compositions comprising the isolated mitochondria provided herein. The compositions, in embodiments, further comprise one or more pharmaceutically acceptable carrier.

In embodiments, the present disclosure provides methods for isolating mitochondria from cells which differs from conventionally known methods and results in mitochondria having the superior functionality and other characteristics provided herein. In embodiments, the method for isolating mitochondria from cells comprises treating cells in a first solution with a surfactant at a concentration below the critical micelle concentration (CMC) for the surfactant, removing the surfactant to form a second solution, incubating the cells in the second solution, and recovering mitochondria from the second solution. In embodiments the concentration of the surfactant in the first solution is about 50% or less of the CMC for the surfactant. For example, in embodiments, the concentration of the surfactant in the first solution is about 40% or less, about 30% or less, about 20% or less, or about 10% or less of the CMC for the surfactant.

In embodiments, the surfactant is a non-ionic surfactant. In embodiments, the surfactant is selected from the group consisting of Triton-X 100, Triton-X 114, Nonidet P-40, n-Dodecyl-D-maltoside, Tween-20, Tween-80, saponin and digitonin. In embodiments, the surfactant is saponin or digitonin. In embodiments, the concentration of the surfactant is less than about 400 μM. For example, in embodiments, the concentration of surfactant in the first solution is less than about 300 μM, less than about 200 μM, less than about 100 μM, or less than about 50 μM. In embodiments, the concentration of the surfactant in the first solution is about 100 μM, about 75 μM, about 60 μM, about 50 μM, about 40 μM, about 30 μM, or about 20 μM. In embodiments, the concentration of the surfactant in the first solution is about 20 μM to about 50 μM, or about 30 μM to about 40 μM.

In embodiments, the first solution further comprises a buffer comprising one or more of a tonicity agent, osmotic modifier, or chelating agent. In embodiments, the first solution comprises a tris buffer, sucrose, and a chelator.

In embodiments, the step of treating the cells in the first solution comprising a low concentration of surfactant (e.g., below the CMC for the surfactant) comprises incubating the cells in the first solution for about 2 minutes to about 30 minutes at room temperature. For example, in embodiments, the step of treating cells in the first solution comprises incubating the cells in the first solution for about 2, about 5, about 10, about 15, about 20, about 25, or about 30 minutes. The incubation may be carried out at a temperature of about 4° C. to about 37° C.

In embodiments, the step of removing the surfactant comprises decreasing the surfactant in the solution to less than 10% of the surfactant concentration in the first solution, or to less than 1% of the surfactant concentration in the first solution. In embodiments, the step of removing the surfactant comprises washing the cells with a buffer.

In embodiments, the step of incubating the second solution comprises incubating the cells in the second solution for about 5 minutes to about 30 minutes. For example, in embodiments, the step of incubating the cells in the second solution comprises incubating the cells in the second solution for about 5, about 10, about 15, about 20, about 25, or about 30 minutes. In embodiments, the step of incubating the cells in the second solution is carried out at a temperature of about 4° C.±3° C. or on ice.

In embodiments, the step of recovering the mitochondria from the second solution comprises collecting the supernatant to recover the isolated mitochondria. In embodiments, the step of recovering the mitochondria from the second solution comprises centrifuging the second solution and collecting the supernatant following centrifugation to recover the isolated mitochondria.

In embodiments, the iMIT may be performed on a cell attaching to a culture surface. In embodiments, the iMIT may be performed on a cell attaching to a culture surface without detaching the cell from the surface. In embodiments, the step of recovering the mitochondria from the second solution comprises collecting the supernatant to recover the isolated mitochondria, which can be optionally followed by washing the remaining cell on the culture surface with the second solution or another second solution to combine it with the supernatant.

In embodiments, the methods provided herein further comprise freezing the isolated mitochondria. In embodiments, the methods comprise freezing the mitochondria in a buffer comprising a cryoprotectant (e.g., glycerol). In embodiments, the methods comprise freezing the mitochondria in the buffer in liquid nitrogen. In embodiments, the methods further comprise thawing the mitochondria after freezing. In embodiments, the methods for thawing the mitochondria comprise rapidly thawing the mitochondria, for example, within about 5 minutes or within about 1 minute. In embodiments, the mitochondria are thawed in a warm bath having a temperature of about 20° C.±3° C. to about 37° C.±3° C. In embodiments, the mitochondria are thawed at a temperature of about 20° C.±3° C. or colder.

In embodiments, the present disclosure provides a population of isolated mitochondria obtained by the method provided herein. In embodiments, the method provided herein is the “iMIT” method and the mitochondria obtained by this method are referred to herein as “Q” mitochondria. In embodiments, the present disclosure provides compositions and/or formulations comprising the population of isolated mitochondria obtained by the methods provided herein.

In embodiments, the present disclosure provides methods for treating or preventing a disease or disorder associated with mitochondrial dysfunction, the method comprising contacting cells of a subject with a population of isolated mitochondria provided herein, e.g., the Q mitochondria. In embodiments, the disease or disorder is an ischemia-related disease or disorder. For example, in embodiments, the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ. In embodiments, the disease or disorder is a genetic disorder. In embodiments, the disease or disorder is a cancer, cardiovascular disease, ocular disorder, otic disorder, autoimmune disease, inflammatory disease, or fibrotic disorder. In embodiments, the disease is acute respiratory distress syndrome (ARDS). In embodiments, the disease or disorder is an aging disease or disorder, or a condition associated with aging. In embodiments, the disease or disorder is pre-eclampsia or intrauterine growth restriction (IUGR).

In embodiments, the present disclosure provides methods for treating or preventing a disease or disorder provided herein, wherein the method comprises administering the population of isolated mitochondria or the composition to a subject in need thereof. In embodiments, the route of administration of the isolated mitochondria is via an intravenous, intra-arterial, intra-tracheal, subcutaneous, intramuscular, inhalation, or intrapulmonary route of administration. In embodiments, the subject is a mammal, e.g., a human.

In embodiments, the present disclosure provides an isolated mitochondrion having intact inner and outer membranes, wherein the inner membrane comprises folded cristae, wherein the mitochondrion has been isolated from a cell, wherein the mitochondrion is polarized as measured by a fluorescence indicator (e.g., JC-1, TMRM, or TMRE), and wherein the mitochondrion is capable of maintaining polarization in an extracellular environment. In embodiments, the folded cristae are densely folded cristae. In embodiments, the mitochondrion has a substantially non-filamentous shape. In embodiments, the mitochondrion comprises voltage dependent anion channels (VDAC) on its surface that are associated with tubulin. For example, in embodiments, the isolated mitochondrion comprises dimeric tubulin associated with VDAC on the surface. In embodiments, the tubulin comprises at least α-tubulin. In embodiments, the tubulin is a heterodimer comprising α-tubulin and β-tubulin. In embodiments, the tubulin is a homodimer. In embodiments, the isolated mitochondrion exhibits decreased association with MAM as measured by GRP75 expression. For example, in embodiments, isolated mitochondrion exhibits about 70%, about 60%, about 50%, about 40%, about 30%, or less association with MAM when compared to mitochondrion that is present in a cell (i.e. has not been isolated), and/or a mitochondrion that has been obtained by a conventional method of isolation such as one involving homogenization and/or high levels of detergent, as further described herein. In embodiments, the isolated mitochondrion provided herein exhibits a decrease in association with MAM, wherein the decrease is at least about 30%, at least about 40%, at least about 50%, at least about 60%, at least about 70%, or more relative to the association with MAM of a mitochondrion that is present in a cell (i.e., has not been isolated) and/a mitochondrion that has been isolated by a conventional method of isolation.

In embodiments, the isolated mitochondrion provided herein has a membrane potential of between about −30 mV and about −220 mV. In embodiments, the isolated mitochondrion is non-filamentous in shape. In embodiments, the isolated mitochondrion is not undergoing drp1-dpendent division. In embodiments, the isolated mitochondrion is between about 500 nm and 3500 nm in size. For example, in embodiments, the isolated mitochondrion is about 500, about 600, about 700, about 800 nm, about 900 nm, about 1000 nm, about 1100 nm, about 1200 nm, about 1500 nm, about 2000 nm, about 2500 nm, about 3000 nm, or about 3500 nm in size.

In embodiments, the present disclosure provides an isolated mitochondrion obtained by the methods provided herein. In embodiments, the present disclosure provides compositions and formulations comprising an isolated mitochondrion provided herein.

### BRIEF DESCRIPTION OF THE DRAWINGS

FIG. 1A shows the distribution of the fluorescence intensity ratio (ratio of mitochondrial fluorescence intensity to background fluorescence intensity) of a fluorescent indicator to mitochondria depolarized with a depolarizing agent. In FIG. 1A, 97.5% of depolarized mitochondria exhibited fluorescence intensities of less than 1.2. Thus, in this experiment, mitochondria were considered to have a membrane potential when the fluorescence intensity ratio exceeded 1.2.

FIG. 1B shows a microscopic image of mitochondria obtained by the method of the present invention by transmitted light and a fluorescence image with a fluorescent indicator (TMRE) in the same region as the transmitted light image. Scale bars indicate 10 mμm.

FIG. 2A shows a TMRE fluorescence image of mitochondria obtained by the DHF method compared to a TMRE fluorescence image of mitochondria obtained by a homogenization method. Scale bars indicate 25 μm.

FIG. 2B, top panel, shows the protein content (μg) of the mitochondrial fraction isolated by the DHF method vs. the homogenization method. FIG. 2B, bottom panel, shows the TMRE positive mitochondria (%) of the mitochondrial fraction isolated by the DHF method vs. the homogenization method. The DHF method resulted in a statistically significantly higher % TMRE positive fraction (p<0.05).

FIG. 2C shows a microscope image (top panels) and TMRE staining fluorescence image (bottom left panel) of isolated mitochondria. The images are merged in the bottom right panel, and shows that almost all mitochondria in the isolated population are polarized.

FIG. 2D shows an electron microscope image of a mitochondrion isolated by the methods provided herein. The scale bar is 1 μm. The image shows that the isolated mitochondrion has intact inner and outer membranes, and densely folded cristae.

FIG. 2E shows STED microscopy images of Q mitochondria, H mito (mitochondria isolated by a conventional homogenization method), and D mito (mitochondria isolated by a conventional detergent method). The outer membranes of the mitochondria were stained green (immunofluorescence of Tom20) and the inner membranes were stained red with Mitotracker Red. A quantification of the ratio of intact outer membranes and the size of isolated mitochondria is provided in Table 3.

FIG. 3A shows the size distribution by dynamic light scattering, polydispersity (PDI), and the measured zeta potential of a population of the mitochondria, prior to freeze-thawing, obtained by mitochondrial extraction without homogenization or without surfactant at concentrations above critical micelle concentrations (DHF method) in the Examples.

FIG. 3B shows the size distribution by dynamic light scattering, polydispersity (PDI), and the measured zeta potential of a population of the mitochondria, after freeze-thawing, obtained by the DHF method.

FIG. 3C shows the size distribution, polydispersity (PDI), and the measured zeta potential of a population of the mitochondrial, after freeze-thawing, obtained by the same method as the DHF method, except that the centrifugation procedure was performed at 1000×g in the DHF method (Item 7 in 2.2.1).

FIG. 3D shows a superimposed image of a transmission microscope image and a TMRE stained image of populations of a pre-freeze-thaw mitochondria and a post-freeze-thaw mitochondria obtained by the DHF method.

FIG. 4 shows the isolation of populations of the mitochondria by DHF method from each of mouse tissues, and the size distribution, polydispersity (PDI), and measured zeta potentials of the populations of the isolated mitochondria by dynamic light scattering.

FIG. 5A shows the TMRE fluorescence intensity over time (minutes) in a single mitochondrion isolated by the iMIT method and treated with 1 mM malate and 1 μM oligomycin at the indicated time points.

FIG. 5B shows TMRE fluorescence images of a single mitochondrion isolated by the iMIT method in a typical time course with malate added at the time points shown in FIG. 5A (1 mM malate was added between minutes 0 and 1, and 1 μM oligomycin was added between minutes 5 and 6).

FIG. 6A shows that isolated mitochondria (middle panels) co-localized with mitochondria in recipient fibroblast cells (left panel). The merged image is shown in the right panel.

FIG. 6B also shows the co-localization of Q mitochondria with mitochondria in recipient cells (bottom panel). In contrast, mitochondria isolated via the homogenized method appear to be inside the recipient cells, but have not co-localized with the endogenous mitochondria.

FIG. 7A shows fixed cells stained to show the shape of mitochondria in the absence (left panel) or presence (right panel) of 10 μM of the Drp1 inhibitor Mdivi1. The mitochondria in the cells maintained the networked, branched shape regardless of the presence or absence of 10 μM Mdivi1.

FIG. 7B. shows that after addition of 30 μM digitonin and a subsequent washing step, the mitochondria in the cells are non-filamentous in shape regardless of the presence or absence of 10 μM Mdivi1.

FIG. 7C shows that the GFP+ mitochondria isolated by iMIT from the cells treated with 0 or 10 μM of Mdivi1 retain the non-filamentous shape.

FIG. 8 shows a comparison in polarization between Q mitochondria (top panels) and mitochondria isolated by a conventional detergent method (middle panels) or conventional homogenization method (bottom panels). The left column of panels is a microscope image and shows that mitochondria were isolated by each of the methods. The right panel shows TMRE fluorescence and indicates that only the Q mitochondria have a high proportion of polarized mitochondria.

FIG. 9A shows calcein fluorescence staining indicating that the Q mitochondria have intact mitochondrial membranes after isolation (top panel), and maintain intact membranes even after addition of 0.96 mM calcium (bottom panel).

FIG. 9B shows TMRE fluorescence staining indicating that the Q mitochondria are polarized before (top panel) and after exposure to calcium (bottom panel).

FIG. 10A shows that physical disruption of the population of mitochondriadisrupts the mitochondrial membrane, and the addition of 0.96 mM calcium further disrupts the membrane such that calcein fluorescence can no longer be detected following the combination of stirring with a high calcium environment.

FIG. 10B shows that membrane potential (measured by TMRE staining) is maintained following physical disruption of the mitochondria by stirring, but is subsequently lost following addition of 0.96 mM calcium.

FIG. 11 shows the GRP75 protein content by western blot assay (left panel) of iMIT mitochondria (Q mitochondria; right lane) compared to mitochondria isolated via a conventional detergent method (D, left lane) or a conventional homogenization method (H, middle lane). Cytochrome oxidase was used as a protein content control (right panel).

FIG. 12 is a schematic providing the study design of the 4 week cardiac infarction model study.

FIG. 13 shows the left ventricle contraction function in rats in the ischemia reperfusion model. EF % is shown for animals in the sham group, the PBS group, the homogenized mitochondria group, and the high and low Q mitochondria groups. **(p<0.01).

FIG. 14 is a schematic providing the study design of the 7 day cardiac infarction model study.

FIG. 15 shows the ejection fraction (EF %) at 1, 3, and 7 days after dosing with PBS or mitochondria (Q) in the test group (IR Q), negative control (IR PBS) or sham infarction (Sham Q) groups.

### DETAILED DESCRIPTION

The present disclosure provides highly functional mitochondria and populations of highly functional mitochondria that are useful in treating a variety of diseases, disorders, and conditions. In embodiments, the mitochondria have been isolated from a cell and retain the capability to function. For example, in embodiments, the mitochondria provided herein have been isolated from a cell and retain a high degree of polarization and/or other aspects of mitochondrial function described herein. The present disclosure further provides methods for obtaining mitochondria such that the obtained mitochondria are highly functional. The methods and isolated mitochondria provided herein are a significant improvement over previously known methods for isolating mitochondria from cells and the isolated mitochondria that resulted from those previously known methods.

As used herein, the terms “isolation” and “isolating” refer to the collection of mitochondria from inside to outside of the cell. The term “isolating” can include removing at least one of the other components in solution from a solution containing mitochondria that have been collected extracellularly. Thus, as used herein, the term “isolated” means that the mitochondria that are no longer within a cell. The terms “processed” or “obtained” and the like may be used interchangeably with “isolated.” In embodiments, an isolated mitochondrion or isolated population of mitochondria has been processed to obtain it from a cellular environment via the methods provided herein. The methods provided herein are a means of obtaining mitochondria from cells in a manner that causes minimal structural damage to the mitochondria and allows them to maintain membrane integrity and membrane potential even after isolation.

As used herein, the term “cell” is a eukaryotic cell, i.e., a cell that contains mitochondria in the cytoplasm, e.g., an animal cell, e.g., a mammalian cell, preferably a human cell. As used herein, the term “cell” is used in the meaning to include a cell present in a tissue, and a cell separated from a tissue (e.g., a single cell), and a cell that is within a population of cells (e.g., a population of cells obtained from a tissue of a subject, and/or a population of cells obtained from a cell line.

As used herein, the term “mitochondrion” is an organelle present in a eukaryotic cell that has double-layered lipid membranes, the inner and outer membranes, and a matrix surrounded by cristae and inner membranes. Mitochondria (more than one mitochondrion) have enzymes on their inner membrane, such as the respiratory chain complexes, which is involved in oxidative phosphorylation. The inner membrane has a membrane potential due to the internal-external proton gradients formed by the action of the respiratory chain complexes, etc. Mitochondria are thought to be unable to maintain the membrane potential when the inner membrane is disrupted. Mitochondria are known to have their own genomes (mitochondrial genomes) that differ from the genome in the cell nucleus. As used herein, a “population of mitochondria” is a population that includes a plurality of mitochondria.

As used herein, the term “polarization” means that the mitochondrion exhibits a membrane potential. As used herein, the term “polarization ratio” is the ratio of polarized mitochondria to total mitochondria. Mitochondrial polarization can be conveniently detected, for example, by commercially available fluorescent indicators, by those skilled in the art. Fluorescent indicators include, without limitation, JC-1, tetramethylrhodamine methyl ester (TMRM), and tetramethylrhodamine ethyl ester (TMRE).

As used herein, the term “surfactant” means a molecule having a hydrophilic moiety and a hydrophobic moiety in one molecule. Surfactants have the role of reducing surface tension at the interface or mixing polar and non-polar substances by forming micelles. Surfactants are roughly classified into nonionic surfactants and ionic surfactants. Nonionic surfactants are those in which the hydrophilic moiety is not ionized, and ionic surfactants are those in which the hydrophilic moiety comprises either a cation or an anion or both a cation and an anion.

As used herein, the term “critical micelle concentration” (CMC) refers to the concentration at which, when the concentration is reached, the surfactant forms micelles, and the surfactant further added to the system contributes to micelle formation, in particular the concentration in bulk. At concentrations above the critical micelle concentration, the addition of surfactants to the system ideally increases the amount of micelles, especially the number of micelles.

Conventional methods for isolating mitochondria have involved methods to mechanically ground (homogenize) the whole cell or using surfactants or detergents to solubilize the cell membrane to collect the mitochondria from the cell. In the latter methods, the surfactant or detergent is administered to the cell at a concentration high enough to disrupt the cell membrane and any membranes within the cell (i.e., at a concentration higher than the CMC). In some cases, these methods (homogenization and use of a high concentration of surfactant or detergent) have been used in combination to increase the yield of mitochondria. Other methods include methods involving freeze-thawing for destruction of cell membrane and/or sonication. Mitochondria obtained by these methods may exhibit some function but considering the low polarization ratio achieved by those methods, it is believed that (1) in the method of homogenizing cells and/or freeze-thawing cells and/or sonicating cells to destroy the cell membrane, the mitochondria that form a network structure within the cell are physically damaged by a membrane-damaging shear stress, and/or ice crystal formation, and/or a membrane-damaging ultrasonic, respectively; and (2) in surfactant-based methods, the mitochondrial membrane is exposed to surfactants, which can solubilize the mitochondria membrane as well as the cell membrane, or the surfactants bind to the mitochondria membrane proteins and then the isolated mitochondria are chemically damaged by the surfactant.

Researchers have also attempted to collect mitochondria by using proteins instead of surfactants to make pores in the plasma membrane⁽¹⁾. This method also yielded some high-quality mitochondria, but the yield was low, and most of the mitochondria were damaged. To increase the yield, the cells were repeatedly pipetted, and the mitochondria outside of the cells seemed to be damaged.

In an aspect, the present disclosure provides mitochondria that have been isolated by a new method, referred to herein as the “detergent and homogenization free (DHF)” method, or alternatively, as the “iMIT” method. As described herein, the mitochondria isolated by the iMIT method are not damaged (e.g., retain inner and outer membrane integrity), and maintain functional capacity (e.g., membrane potential). The mitochondria obtained by the iMIT method are referred to herein as “Q” mitochondria. These mitochondria are suitable for use in treatments for various diseases and disorders including those described herein, e.g., by mitochondrial transplantation. Mitochondrial transplantation is a treatment that is expected to have a utility in a variety of diseases and disorders. Exogenous mitochondria (e.g., Q mitochondria) are internalized into cells in which mitochondria are severely dysfunctional and/or cells in which an influx of highly functional mitochondria is a benefit, to restore and/or enhance mitochondrial function.

Another application contemplated herein is to study mitochondrial mechanisms, especially mitochondrial responses to stimuli. Isolated mitochondria will be well suited for investigating how mitochondria respond to intracellular signals because of their controllability of their surrounding environment (e.g., the demonstration of Peter Mitchell's chemiosmotic theory that a proton-motive force was responsible for driving the synthesis of ATP, i.e., protons are pumped across the inner mitochondrial membrane as electrons go through the electron transfer chain, may be carried out in isolated mitochondria). Considering the polarization ratio of mitochondria obtained by the present method, it is believed that isolated mitochondria obtained by the conventional method suffer severe damage to both outer and inner membranes. Therefore, by collecting a large number of mitochondria by conventional methods, it is believed that only a small portion of the remaining function is measured. In contrast, mitochondria obtained by the methods of the present disclosure will enable measurement of many phenomena that are not measurable in damaged mitochondria.

Contamination of damaged mitochondria can cause adverse effects on living organisms and cells. The mitochondria provided herein are superior to those isolated by conventional methods in part because they are associated with no cytotoxicity, and/or far less cytotoxicity when compared, for example, with mitochondria isolated via conventional methods. Moreover, the mitochondria provided herein are superior to those isolated by conventional methods because they are superior in functional capacity as described herein. In the method of the present disclosure, it is expected that the extent of damage can be reduced because the mitochondria are free from the effects of physical disruption or chemical destruction by surfactants during their collection process, come in contact with no surfactants or the surfactant at a concentration far below the CMC that cannot be removed from the first solution and cannot damage the mitochondria during the collection processes of iMIT, and that the extent of damage can be minimized, particularly if they are in contact with no surfactants, and thus, it can be expected to reduce the adverse effects of damaged mitochondria on the organism and cells.

### Methods of Obtaining Mitochondria

In embodiments, the present disclosure provides methods for recovering or isolating mitochondria from cells by treating cells in solution with a surfactant at a concentration below the critical micellar concentration (CMC), removing the surfactant from the solution containing the treated cells, and then incubating the surfactant-treated cells to recover the mitochondria into the solution, thereby recovering the mitochondria from the cells. The method is referred to herein as “iMIT”. Accordingly, provided herein is iMIT, a method for obtaining mitochondria from a cell, comprising:

(A) treating cells with a surfactant at a concentration below the critical micelle concentration (CMC) in a first solution,

(B) removing the surfactants from the first solution to form a second solution, and

(C) incubating the surfactant-treated cells in the second solution to recover mitochondria in the second solution. Additional configurations of (A) to (C) above and of the present method are described below.

According to the method of the present disclosure, cells having mitochondria in their cytoplasm are treated with a surfactant at a concentration below the critical micelle concentration in solution. Thus, in embodiments, the cell membranes are weakened in structural strength but are not permeabilized because of the low concentration of the surfactant, while the mitochondrial membranes are exposed to little or no surfactant and remain intact. In embodiments, the cell membranes may be partially permeabilized, but the mitochondrial membranes are exposed to little or no surfactant due to the low concentration of the surfactant and remain intact.

In embodiments, the solution of (A) may comprise a buffer. Exemplary buffers for use in the methods provided herein include, for example, Tris buffer, HEPES buffer, and phosphate buffer. Buffers may be, for example, pH 6.7-7.6 (e.g., pH 6.8-7.4, pH 7.0-7.4, e.g., pH 7.2-7.4, e.g., pH 7.4). In embodiments, the buffers may include tonicity agents and osmotic modifiers. Exemplary tonicity agents and osmotic modifiers include monosaccharides (e.g., glucose, galactose, mannose, fructose, inositol, ribose, xylose, etc.), disaccharides (e.g., lactose, sucrose, cellobiose, trehalose, maltose, etc.), trisaccharides (e.g., raffinose, melesinose, etc.), polysaccharides (e.g., cyclodextrin, etc.), sugar alcohols (e.g., erythritol, xylitol, sorbitol, mannitol, maltitol, etc.), glycerin, diglycerin, polyglycerin, propyleneglycol, polypropyleneglycol, ethyleneglycol, diethyleneglycol, triethyleneglycol, polyethyleneglycol, and the like. Buffers may also contain a chelating agent, particularly a chelating agent for divalent metals, such as a chelating agent for calcium ion. Chelating agents include, for example, glycol ether diaminetetraacetic acid (EGTA) and ethylenediaminetetraacetic acid (EDTA).

In embodiments, a buffer may be a Tris buffer comprising sucrose and a chelator, wherein the pH is 6.7-7.6 (e.g., pH 6.8-7.4, pH 7.0-7.4, e.g., pH 7.2-7.4, e.g., pH 7.4). In embodiments, the Tris buffer may comprise digitonin or saponin, or another surfactant provided herein. In embodiments, the digitonin or saponin or other surfactant may have a concentration of 20% or less, 15% or less, 14% or less, 13% or less, 12% or less, 11% or less, or 10% or less of the critical micelle concentration. In embodiments, digitonin may be used at concentrations of 400 μM or less, 350 μM or less, 200 μM or less, 150 μM or less, 100 μM or less, 90 μM or less, 80 μM or less, 70 μM or less, 60 μM or less, 50 μM or less, 40 μM or less, or 30 μM or less (e.g., at a concentration of 30 μM). In embodiments, saponins may be used at concentrations of 400 μM or less, 400 μM or less, 350 μM or less, 200 μM or less, 150 μM or less, 100 μM or less, 90 μM or less, 80 μM or less, 70 μM or less, 60 μM or less, 50 μM or less, 40 μM or less, or 30 μM or less (e.g., at a concentration of 30 μM).

In embodiments, the surfactant used in the methods provided herein may be an ionic or a nonionic surfactant. Nonionic surfactants used in the present invention may include, for example, ester, ether, and alkyl glycoside forms. Non-ionic surfactants include, for example, alkyl polyethylene glycols, polyoxyethylene alkylphenyl ethers, and alkyl glycosides. Nonionic surfactants may include Triton-X 100, Triton-X 114, Nonidet P-40, n-Dodecyl-D-maltoside, Tween-20, Tween-80, saponin and/or digitonin. In the treating step (A), at least one of the surfactants selected from the group consisting of Triton-X 100, saponin and digitonin is used. In embodiments, the surfactant is saponin or digitonin.

In embodiments, the treatment step (A), comprises treating the cells with a surfactant at a concentration below the critical micelle concentration. The treatment time of the cells in step (A) may be, for example, 1-30 minutes, for example, 1-10 minutes, or for example, 1-5 minutes, for example, 2-4 minutes, for example, 3 minutes. The treatment of the cells in (A) may be carried out on ice, at 4° C. or at room temperature, or at a temperature between.

In embodiments, the concentration of surfactant in the treatment step (A) can be at a concentration below the critical micelle concentration, e.g., 90% or less, 80% or less, 70% or less,60% or less, 50% or less, 40% or less, 30% or less, 20% or less, 15% or less, 14% or less, 13% or less, 12% or less, 11% or less, 10% or less, for example, 5-15%, for example, 8-12%, for example 10% of the critical micelle concentration.

In embodiments, the treatment step (A) is a pretreatment of the cells. Without wishing to be bound by theory, it is believed that treatment of the cells with a surfactant below the critical micelle concentration can reduce the strength of the cell membrane; and/or partially or completely eliminates the effect of detergents on intracellular mitochondria.

Thus, in view of minimizing the effect of surfactants on mitochondria, the concentration of surfactant in the solution in which the mitochondria come into contact at least in any step (e.g., each of steps (B) to (E)) during and after recovering the mitochondria from the cell can be below the critical micelle concentration, e.g., less than 10%, less than 5%, less than 4%, less than 3%, less than 2%, or less than 1% or less of the critical micelle concentration; or below the detection limit. In view of minimizing the effects of surfactants on mitochondria, it is preferred that no surfactant should be added to the solution in which the mitochondria come into contact, during and after recovering the mitochondria from the cell.

In embodiments, the cells may be in the form of cells present in a tissue, or they may be isolated from a tissue (e.g., single cells) or a population thereof. The cells isolated from the tissue may be cultured cells, or single cells or a population thereof, obtained by treatment of the tissue or cultured cells with enzymes used to make them be single cells, such as collagenase. Tissues may be chopped, if desired, prior to enzymatic treatment, such as collagenase.

In embodiments, the surfactant can be removed from the solution before mitochondria are recovered from the surfactant-treated cells in (A) in order to reduce the concentration of surfactant in contact with the mitochondria or to sufficiently reduce the surfactant in contact with the mitochondria.

In the removing step (B), removal of surfactants can be performed, for example, by replacing the buffer with a solution containing a lower or reduced concentration of surfactant (preferably a surfactant-free solution) (e.g., a buffer) or adding the solution to the buffer. If the surfactant-treated cells are adherent cells, the buffer containing the surfactant can be removed by aspirating the solution, rinsing the cells in a solution containing a lower or reduced concentration of surfactant (preferably a surfactant-free solution) (e.g., a buffer) if needed, and adding a solution containing a lower or reduced concentration of surfactant (preferably a surfactant-free solution) (e.g., a buffer). If the surfactant-treated cells are floating cells, it is possible to remove the surfactant by centrifuging the cells, removing the supernatant, rinsing the cells in a solution containing a lower or reduced concentration of surfactant (preferably a surfactant-free solution) (e.g., a buffer) if needed, and adding a solution containing a lower or reduced concentration of surfactant (preferably a surfactant-free solution) (e.g., a buffer).

Removal means at least decreasing the concentration of surfactant in the solution in which the mitochondria come into contact, including, for example, less than 10%, less than 5%, less than 4%, less than 3%, less than 2%, or less than 1% or less of the concentration of surfactant; or below the detection limit in the solution in which the mitochondria come into contact. To ensure removal of the surfactant from the solution, (B) may include washing the cells with a solution containing a lower or reduced concentration of surfactant (preferably a surfactant-free solution) (e.g., a buffer).

In (B), in order to remove the surfactant from the solution, the solution added to or exchanged with the solution may preferably be a buffer and may be a buffer as described in (A) above (but a solution containing a lower concentration of surfactant, preferably a solution with no surfactant or undetectable levels of surfactant).

Cells treated in (A) have a reduced plasma membrane strength and can allow mitochondria to be released from the cell interior to the extracellular area merely by incubating them in a solution. However, in the steps before (C), the amount of surfactant contacting the mitochondrion is small, and the effect of surfactant on the mitochondrion is limited, thus the decrease in the intensity of the mitochondrial membrane is also limited and/or the mitochondrial membranes remain intact.

In embodiments, the method comprises obtaining mitochondria that are released into the second solution simply by allowing the cell to stand still in the second solution.

Thus, in the present invention, the surfactant-treated cells can be incubated in solution to release mitochondria from the cell interior to the extracellular area. The term “release” in (C) means that mitochondria exit from the interior of the cell to the outside of the region surrounded by the plasma membrane (e.g., on the solution side or extracellular side).

The solution for use in incubating in (C) (the “second solution”) may be a solution containing a lower concentration of surfactant. In preferred embodiments, the second solution is a surfactant-free solution or a solution with a negligible and/or undetectable amount of surfactant. Solutions for use in incubating in (C) may be, for example, buffers as described in (A) above and may be buffers (with lower concentrations of surfactants than one as described in (A) above (preferably surfactant-free solutions). The solution used in (C) may be a solution comprising, for example, a buffer, an osmotic modifier, and a divalent metal chelator, substantially free of surfactants. As used herein, “substantially free” is used in the sense of not excluding the presence of contamination with an amount of “substantially free ingredient” that cannot be removed or cannot be detected.

In (C), the incubation may be, for example, 1-30 minutes, for example, 5-25 minutes, or for example, 5-20 minutes, for example, 5-15 minutes, for example, 10 minutes. The treatment of the cells in (C) may be carried out on ice, or at room temperature, or at a temperature between them.

In (C), a physical stimulus can be added such that the lipid bilayer of the mitochondrion does not cause mechanical disruption, in order to enhance the recovery of the mitochondria from the cell. Thus, in (C), for example, the incubation can be carried out under shaking or non-shaking conditions. In (C), for example, the incubation can be carried out under stirring or non-stirring conditions. In (C), surfactant treatment makes the cells easier to detach from the adhesive surface, so detachment of the cells from the adhesive surface by mild water flow as described above does not appear to negatively affect the polarization ratio. Alternatively, in (C), the incubation can be carried out to the extent that the cells will not become detached.

In (C), mitochondria recovered in solution can be used in various applications as isolated mitochondrial populations. In embodiments, the present disclosure provides populations of mitochondria produced via the method provided herein, which are referred to herein as “Q” mitochondria. In embodiments, the present disclosure provides individual mitochondrion produced via the method provided herein (i.e., individual Q).

In embodiments, the methods provided herein further comprise (D) purifying mitochondria recovered in solution. Mitochondria can be separated from one or more other cellular components by centrifugation. For example, mitochondria can be purified as supernatants by centrifugation of the mitochondrial population recovered in (C) at 1500 g or less, 1000 g or less, or 500 g or less to precipitate contaminants such as the detached cells contained in the mitochondrial population. The mitochondria can preferably be purified, for example, as supernatants by centrifugation at 500 g. Mitochondria may also be collected as a precipitate by subjecting the resultant supernatant to further centrifugation (e.g., 8000 g to 12000 g) for enrichment and the like. The term “purified” used herein means that the mitochondria are separated from at least one of the other components in solution by the manipulation.

The mitochondrial population obtained in (C) and/or (D) above can be used as an isolated mitochondrial population in various applications.

The method of the present invention may further comprise (E) freezing mitochondria. Freezing can be performed by mildly suspending the mitochondria in a buffer for freezing. The buffer for freezing may be a buffer as described in (A), but not including a surfactant, and may further comprise a cryoprotectant. Exemplary cryoprotectants are known in the art and include, for example, glycerol, sucrose, trehalose, dimethyl sulfoxide (DMSO), ethylene glycol, propylene glycol, diethyl glycol, triethylene glycol, glycerol-3-phosphate, proline, sorbitol, formamide, and polymers. Thus, the mitochondria provided herein can be stored by freezing. In the method of the present disclosure, mitochondria may not be frozen if cryopreservation is not necessary, e.g., the mitochondria may be used when freshly isolated. In other embodiments, the mitochondria may be stored at 4° C.±3° C. or on ice. In embodiments, the mitochondria provided herein produced by the method provided herein may be stored in liquid nitrogen, at about −80° C.±3° C. or lower, about −20° C.±3° C. or lower, or about 4° C.±3° C. In embodiments, the mitochondria may be stored for days, weeks, or months, or longer, and retain the capacity to function after thawing.

In embodiments, the methods provided herein further comprise methods for thawing the mitochondria that have been isolated as provided herein and subsequently frozen. Methods for thawing the mitochondria provided herein comprise thawing the mitochondria at a temperature of about 20° C.±3° C. or colder, and thawing the mitochondria rapidly, for example, within about 5, about 4, about 3, about 2, or about 1 minute. In embodiments, the rapid thaw of the mitochondria results in the mitochondria retaining the functional capabilities described herein.

In embodiments, the methods provided herein do not comprise methods of disrupting the cell membrane in the whole process of collecting mitochondria from a cell in such a manner that the mitochondrial membranes are disrupted. For example, in the methods provided herein, the cells are not disrupted by homogenization during the process of collecting mitochondria from cell. That is, in embodiments, the methods provided herein do not comprise homogenization; in embodiments, the methods comprise homogenization but the homogenization is carried only to the extent that it does not cause any bubbles or bubbles to the solution relative to the cell or tissue. In embodiments, the methods also do not comprise freeze-thawing of cells. Although repeated freeze-thawing of cells is suitable for disrupting the plasma membrane and recovering its contents, and can be used to retrieve mitochondria from the cell, freeze-thawing is believed to also disrupt the mitochondrial lipid bilayer because the membrane potential of the obtained mitochondria is not maintained (as opposed to the method of the present disclosure, in which the mitochondrial membrane potential is maintained).

In embodiments, the methods of the present disclosure do not include other methods of disrupting the cell membrane (e.g., sonication, treatment with a strong stream of water to the extent that a solution produces bubbles, or to the extent that the solution foams) during the whole process of collecting mitochondria from cell. In embodiments, the method of the present disclosure is performed without performing any processes that may substantially cause physical, chemical, or physiological damage to the mitochondria, although a freeze-thaw cycle can be applied to the mitochondria for storage. Thus, the method of the present invention is capable of obtaining mitochondria with minimal damage.

The method of the present invention does not require one or more filtration steps in purifying mitochondria recovered from cells.

In embodiments, the methods provided herein gently separate the mitochondria from the microtubule system without damage to the mitochondria, while the mitochondria are still in the cell. During the incubation period, the mitochondria, which have become non-filamentous in shape due to the detachment of the microtubules from the mitochondrial surface, are able to exit the cell through the surfactant-treated cell membrane. Thus, the mitochondria obtained from the cell via the disclosed method are obtained without ripping and tearing of the mitochondrial membrane or otherwise damaging the structure of the mitochondria. Thus, the isolated mitochondria and populations thereof provided herein are capable of maintaining function after isolation and are vastly more suitable for use in treating disease conditions than any previously described isolated mitochondria.

Accordingly, the methods provided herein differ from conventional methods for isolating mitochondria in important ways, and provide isolated or obtained mitochondria that have surprising and advantageous features relative to mitochondria isolated by conventional methods or any other previously disclosed method.

### Population of Mitochondria

In an aspect, the present disclosure provides populations of mitochondria that have been isolated from a cell using the methods provided herein and as a result, are highly functional. As described above, the novel method of isolation provided herein is referred to interchangeably as the “DHF” method or the “iMIT” method; the mitochondria obtained via the DHF or iMIT method is referred to herein as “Q” mitochondria. The Q mitochondria have been spared from the disruption and membrane destruction that occurs when mitochondria are isolated via conventional methods, and thus are structurally and functionally superior to mitochondria isolated via conventional methods.

In embodiments, the present disclosure provides a population of isolated or obtained mitochondria, wherein the population contains a high proportion of polarized mitochondria (i.e., the population has a high polarization ratio). Thus, the population of mitochondria provided herein comprises a high proportion of mitochondria having membrane potential. In embodiments, the present disclosure provides a population of mitochondria, wherein a high proportion of the mitochondria in the population have intact inner and outer membranes. In embodiments, the presence of intact inner and outer membranes can be determined by the functional activity of the mitochondria, for example, the membrane potential and polarization.

The population of mitochondria provided herein is thus superior from mitochondrial populations obtained from cells using conventional methods, such as methods that involve homogenization and/or freeze-thaw of cells and/or high concentrations of detergents or surfactants, as described above. For example, the mitochondria isolated from cells via conventional methods are necessarily damaged by the isolation process and lose functional capacity. Accordingly, in the present disclosure provides a population of isolated mitochondria having a higher polarization ratio and/or a higher % polarization and/or higher % mitochondria with an intact inner and outer membrane, than a population of mitochondria obtained by conventional methods.

In embodiments, the polarization ratio of the population of isolated or obtained mitochondria may be, for example, 40% or more, 45% or more, 50% or more, 55% or more, 60% or more, 65% or more, 70% or more, 75% or more, 80% or more, or 85% or more.

In embodiments, at least about 60%, at least about 65%, at least about 70%, at least about 75%, at least about 80%, at least about 85%, at least about 90%, at least about 95%, or more of the population of isolated or obtained mitochondria are polarized as measured by a fluorescence indicator. In embodiments, the fluorescence indicator may be any fluorescence indicator known to the person of ordinary skill in the art to be suitable for measuring mitochondrial membrane potential. In embodiments, the fluorescence indicator is selected from the group consisting of JC-1, TMRM, and TMRE.

In embodiments, at least about 60%, at least about 65%, at least about 70%, at least about 75%, at least about 80%, at least about 85%, at least about 90%, at least about 95%, or more of the population of isolated or obtained mitochondria have intact inner and outer membranes. In embodiments, at least about 60%, at least about 65%, at least about 70%, at least about 75%, at least about 80%, at least about 85%, at least about 90%, at least about 95%, or more of the population of isolated or obtained mitochondria have densely folded cristae in the inner membrane. For example, in embodiments the cristae structure of the Q mitochondria resembles that of the cristae structure of mitochondria that are in a cell, i.e., has not been isolated from a cell. The term “densely folded cristae” as used herein means that the mitochondria comprise cristae present at a high density, that is, highly folded cristae. The density of cristae may be assessed using microscopy (e.g., transmission electron or optical microscopy including confocal microscopy). In embodiments, cristae density in mitochondria may be measured by the number of cristae folds per square micrometer, which can be manually determined by counting the number of folds and/or via an automated software program. In embodiments, “high density of cristae,” “densely folded cristae,” and the like means at least about 3, at least about 4, at least about 5, at least about 6, at least about 7, at least about 8, or more cristae (i.e., cristae folds) per square micrometer. Alternatively or additionally, cristae density in mitochondria may be measured by the cristae surface area per mitochondrial volume. Thus, in embodiments, “high density of cristae,” “densely folded cristae,” and the like means that the cristae surface area per mitochondrial volume (μm² μm⁻³) is at least about 20, at least about 25, at least about 30, at least about 35, at least about 40, or more. Methods for determining cristae density are known in the art (see, for example, Segawa et al., “Quantification of cristae architecture reveals time dependent characteristics of individual mitochondria” Life Science Alliance vol. 3 no. 7, June 2020; and Nielsen et al., The Journal of Physiology 595.9 (2017) pp. 2839-47). In embodiments, at least about 60%, at least about 65%, at least about 70%, at least about 75%, at least about 80%, at least about 85%, at least about 90%, at least about 95%, or more of the mitochondria in the population of mitochondria provided herein have at least about 3, at least about 4, at least about 5, at least about 6, at least about 7, at least about 8, or more cristae per square micrometer; and/or have at least about 20 cristae surface area per mitochondrial volume (μm² μm⁻³), at least about 25 μm² μm⁻³, at least about 30 μm² μm⁻³, at least about 35 μm² μm⁻³, at least about 40 μm² μm⁻³, or more. In embodiments, the isolated mitochondria provided herein have average or representative cristae density that is equivalent to and/or not significantly less than the cristae density of mitochondria in the cell type from which the isolated mitochondria were obtained. In embodiments, at least about 60%, at least about 65%, at least about 70%, at least about 75%, at least about 80%, at least about 85%, at least about 90%, at least about 95%, or more of the mitochondria in the population of mitochondria provided herein exhibit cristae density that is equivalent to and/or not significantly less than the average or representative cristae density of mitochondria in the cell type from which the isolated mitochondria were obtained.

In embodiments, the population of isolated mitochondria provided herein have the surprising feature of maintaining functional capability even when exposed to a high calcium (Ca²⁺) environment. In embodiments, the population of isolated mitochondria provided herein maintain functional capability in an extracellular environment due to the methods of isolation provided herein. In embodiments, at least about 60%, at least about 65%, at least about 70%, at least about 75%, at least about 80%, at least about 85%, at least about 90%, at least about 95%, or more of the population of isolated or obtained mitochondria maintain functional capability in an extracellular environment. In embodiments, the extracellular environment comprises a total calcium concentration of about 6 mg/dL to about 14 mg/dL, or of about 8 mg/dL to about 12 mg/dL. In embodiments, the extracellular environment comprises concentration of free/active calcium of about 3 mg/dL to about 8 mg/dL, or of about 4 mg/dL to about 6 mg/dL. Thus, in embodiments, the Q mitochondria provided herein possess the remarkable characteristics of being isolated from a cellular environment with minimal or negligible damage, and retain capacity to function even when exposed to an extracellular environment, e.g., a calcium rich environment that would otherwise be expected to cause damage to the mitochondria and/or significantly inhibit their functional capacity.

Without wishing to be bound by theory, in some embodiments, the ability of the isolated or obtained mitochondria provided herein to maintain functional capability in an extracellular environment is due, in part or in whole, to the association of tubulin with voltage dependent anion channels (VDAC) on the mitochondrial surface. For example, in embodiments, during the iMIT isolation process provided herein, tubulin may associate with all or a substantial number of VDAC on the mitochondrial surface such that mitochondria are capable of maintaining function even in a calcium rich environment (e.g., an extracellular environment comprising about 3 mg/dL to about 14 mg/dL calcium, or more). In embodiments, the association of tubulin with VDAC on the surface of the isolated mitochondria may be determined by detecting the presence of tubulin at the mitochondrial surface, for example by staining.

Without wishing to be bound by theory, in some embodiments, the isolated Q mitochondria provided herein are able to maintain functional capability in an extracellular environment due, in whole or in part, to a depletion of cholesterol, ergosterol, and/or related molecules in the outer membrane of the Q mitochondria during iMIT isolation. That is, cholesterol (which stabilizes VDAC structure) may be depleted to an extent due to contact of a small amount of surfactant with the mitochondrial membrane during the isolation procedure, resulting in isolated mitochondria having VDAC on the surface that have lost some or all function, such that the mitochondria become resistant to extracellular calcium concentrations (e.g., an extracellular environment comprising about 3 mg/dL to about 14 mg/dL calcium, or more). Thus, in embodiments, the isolated mitochondria provided herein comprise a very low level of sterol concentration in the mitochondrial membrane.

In embodiments, the population of isolated or obtained mitochondria further exhibit reduced association with mitochondria-associated membrane (MAM) relative to mitochondria that are in a cell and/or mitochondria that have been isolated or obtained using a conventional method such as one that involves homogenization of cells and/or freeze thaw of cells. In embodiments, the decreased association with MAM is measured by glucose regulated protein GRP75 expression at the surface of the mitochondria.

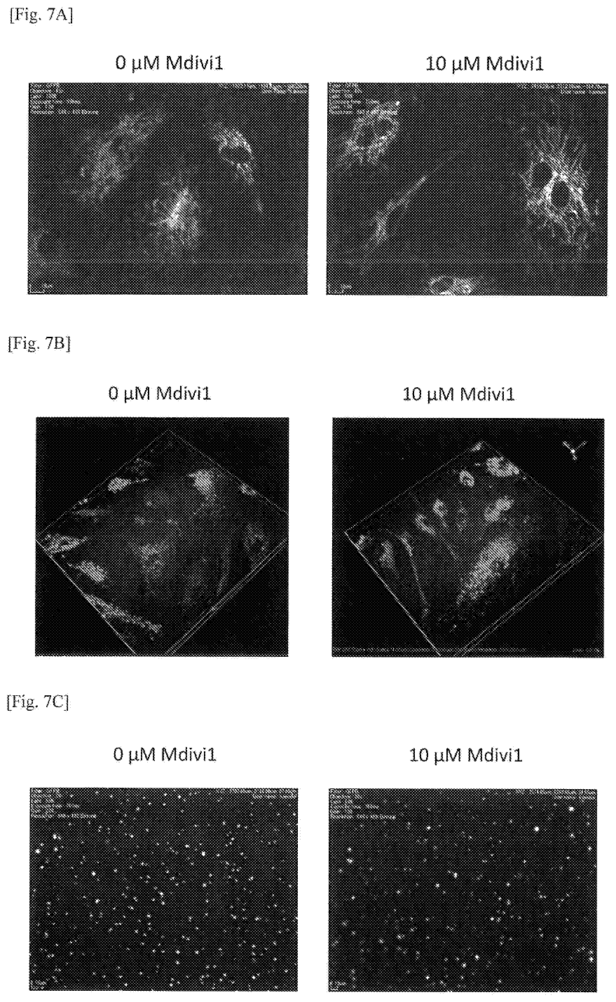

In embodiments, the isolated mitochondria are substantially non-filamentous in shape. “Non-filamentous” may be used interchangeably with “non-network-like” and the like, and means that the mitochondria do not exhibit the branched and mesh-like network of mitochondria that exist within a cell (see, for example, representative filamentous shape of mitochondria in cells in FIG. 7A). In embodiments, rather than having a filamentous, networked, or branched structure, the mitochondria provided herein appear as round, globular, irregularly shaped, and/or slightly elongated, or any mixture thereof, when viewed under a microscope. At lower magnitude, the isolated mitochondria appear as a dot-like structure. In contrast, at lower magnitude, the highly elongated, network, or branched structure of mitochondria in a cell is visible. In embodiments, at least about 60%, at least about 65%, at least about 70%, at least about 75%, at least about 80%, at least about 85%, at least about 90%, at least about 95%, or more of the population of isolated or obtained mitochondria have a long diameter to short diameter ratio of no more than 4:1, no more than 3.5:1, or no more than 3:1. Without wishing to be bound by theory, the shape of the mitochondria isolated via the methods provided herein results from the gentle removal of connections via motor proteins to microtubules while the mitochondria is still in the cell prior to isolation. That is, once the mitochondria are no longer tethered to the microtubules of the cell, they lose the highly elongated and branched/networked shape that they had within the cell to instead form the non-filamentous shape described herein.

In embodiments, at least about 60%, at least about 65%, at least about 70%, at least about 75%, at least about 80%, at least about 85%, at least about 90%, or at least about 95% of the isolated mitochondria in the population of mitochondria provided herein have a length shorter than the double of the hydrodynamic diameter of the mitochondria. In embodiments, the hydrodynamic diameter is about 1 μm, and at least about 60%, at least about 65%, at least about 70%, at least about 75%, at least about 80%, at least about 85%, at least about 90%, or at least about 95% of the isolated mitochondria in the population of mitochondria provided herein have a length of 2 μm or less, 1.9 μm or less, 1.8 μm or less, 1.7 μm or less, 1.6 μm or less, 1.5 μm or less, 1.4 μm or less, or 1.3 μm or less in length of the major axis. In embodiments, hydrodynamic diameter is measured by Dynamic Light Scattering method (DLS). In embodiments, hydrodynamic diameter is a median diameter D₅₀.

In general in a cell, mitochondria are highly elongated in shape or in the form of filamentous, branched structures as described above. Non-filamentous and non-elongated mitochondria generally only exist in a cell when drp1-dependent division, or drp1-dependent fission, is occurring. For this process of mitochondrial fission, interaction with the endoplasmic reticulum causes initial constriction of the mitochondrion. Drp1 proteins are recruited to mitochondria and assemble on its surface to cause further constriction. DYN2 is recruited to carry out the final stage of membrane scission. The resulting mitochondria may generally be spherical in shape. In a cell, such spherical mitochondria may retain spherical shape for a limited period of time before becoming elongated or forming the more typical branch-like structures. In contrast, mitochondria isolated using the iMIT method are non-filamentous in shape without undergoing Drp1 mediated fission. In addition, mitochondria isolated by conventional methods such as methods involving homogenization of a cell yield mitochondria that are non-filamentous and largely rounded or spherical in shape because they have been damaged and torn from the microtubules in the cell that otherwise cause them to maintain an elongated shape. In contrast to mitochondria isolated in such a manner, the mitochondria isolated by the iMIT method provided herein have not undergone damaging removal from microtubules and are not undergoing drp1 mediated fission. Accordingly, the mitochondria of the present disclosure differ from both natural mitochondria in a cell and mitochondria isolated by more conventional methods. For example, in embodiments, the mitochondria provided herein, obtained via the iMIT method, are substantially non-filamentous in shape while at the same time exhibiting a highly functional status (e.g., polarization), intact inner and outer membrane structure including densely folded cristae, and while not undergoing drp1 fission.

In embodiments, the Q mitochondria provided herein, when contacted with a cell or with a population of cells, exhibit the surprising feature of co-localization with endogenous mitochondria within the cell or cells. The Q mitochondria co-localize with the endogenous mitochondria to a much higher degree compared to mitochondria isolated via conventional methods. In embodiments, the Q mitochondria provided herein, when contacted with a cell or with a population of cells, fuse with endogenous mitochondria within the cell or cells. The fusion of the isolated Q mitochondria is a distinct difference from, and advantage over, mitochondria isolated via conventional methods. In embodiments, the mitochondria retain this ability even after storage. Thus, in embodiments, the Q mitochondria provided herein are superior to conventionally isolated mitochondria at least in that they are more efficient at co-localization with and/or fusion with endogenous mitochondria in cells and thus exhibit a superior clinical effect when used to treat any diseases or disorders such as those described herein. This may suggest that the Q mitochondria provided herein have more robust and nearly intact outer membrane, compared to conventionally isolated mitochondria.

In embodiments, the present disclosure provides a population of mitochondria that is isolated or obtained by the methods provided herein. For example, the present disclosure provides a population of mitochondria that is isolated or obtained by a method comprising steps (A) to (C) of the iMIT method as herein described above. In embodiments, the present disclosure provides a population of mitochondria that is isolated or obtained by a method comprising the steps of (A) to (E) as herein described above.

According to the present disclosure, there is provided a composition comprising a population of isolated mitochondria of the present invention. According to the present disclosure, there is provided a mitochondrial formulation comprising a population of isolated mitochondria of the present invention. Compositions comprising a population of isolated mitochondria of the present invention may further comprise a buffer. Mitochondrial formulations comprising a population of isolated mitochondria of the present invention are pharmaceutically acceptable and may further comprise pharmaceutically acceptable additional components, e.g., excipients. A population of isolated mitochondria of the disclosure, or compositions or mitochondrial formulations containing it, may be obtained during the separation process without using cell sorting by flow cytometer such as fluorescence activated cell sorting (FACS). Thus, a population of isolated mitochondria of the present invention, or a composition or mitochondrial preparation containing it, does not contain fluorescent dyes and fluorescent probes (as well as non-fluorescent mitochondrial stains and probes). In embodiments, the composition is a pharmaceutical composition.

### Detection of Mitochondrial Membrane Potential

Whether a mitochondrion has a membrane potential or not (polarized or not) can be determined by detecting the mitochondrial membrane potential. The mitochondrial membrane potential can be detected using an indicator, e.g., a fluorescent indicator. Fluorescence indicators that detect mitochondrial membrane potentials include JC-1, tetramethylrhodamine methyl ester (TMRM), and tetramethylrhodamine ethyl ester (TMRE). JC-1 accumulates in mitochondria, sensing mitochondrial membrane potential and turning green to red. TMRM and TMRE accumulate in mitochondria, sensing mitochondrial membrane potential and producing red light.

Depolarized mitochondria may be used as a negative control upon detecting mitochondrial membrane potential. Mitochondria can be depolarized by mitochondrial depolarizing agents. Mitochondrial depolarizing agents include, for example, carbonyl cyanide-m-chlorophenyl hydrazone (CCCP). For example, a mitochondrial membrane potential (or fluorescence intensity with a fluorescent indicator) after depolarized by incubation for 1 hour at room temperature in the presence of 5 μM CCCP can be used as a negative control, and mitochondria that have potential (or fluorescence intensity with a fluorescent indicator) above the membrane potential of the negative control can be determined to be mitochondria having membrane potential. Fluorescence intensities of the analyte and the negative control may be determined (e.g., as a ratio to or difference from background fluorescence intensity) by excluding the influence of fluorescence from the background. When the mitochondrial membrane potential of the negative control varies, for example, mitochondria having membrane potential larger than the membrane potential that 90% or greater, 91% or greater, 92% or greater, 93% or greater, 94% or greater, 95% or greater, 96% or greater, 97% or greater, 98% or greater, or 99% or greater of the negative control have can be determined to be mitochondria having membrane potential. In this way, the mitochondrial membrane potential in a population of mitochondria can be detected. In the method of the present disclosure, no additional steps that lead to loss of the mitochondrial membrane potential can be performed in the detection of the mitochondrial membrane potential.

Mitochondrial polarization ratio is the ratio (%) of the number of mitochondria having a membrane potential to the total number of mitochondria. Mitochondrial polarization ratio can be calculated from, for example, the number of mitochondria contained in a certain region of the substrate board such as glass (e.g., 100 μm² to 10,000 μm²) to which the mitochondria are immobilized, and the number of mitochondria having membrane potentials within the region. Mitochondria can be counted, for example, using a light microscope.

### Methods of Treatment

In one aspect, the present disclosure provides methods for treating diseases and disorders associated with mitochondrial dysfunction or diseases or disorders that otherwise benefit from the supplementation of healthy, functional mitochondria.

In embodiments, the disease or disorder suitable for treatment with the Q mitochondria provided herein is a genetic disease or disorder, an ischemia related disease or disorder, a neurodegenerative disease or disorder, a cancer, a cardiovascular disease or disorder, an autoimmune disease, an inflammatory disease, a fibrotic disease, an aging disease or disorder, or a disease or associated with complications of birth.

Exemplary ischemia-related diseases and disorders include cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing (e.g., an acute wound or a chronic wound; a cut, laceration, compression wound, burn wound (e.g., chemical, heat or flame, wind, or sun burn), or a wound resulting from a medical or surgical intervention), spinal cord injury, sickle cell disease, and reperfusion injury of a transplanted organ. In embodiments, the Q mitochondria may treat, prevent, ameliorate, and/or improve clinical condition due to ischemia-reperfusion injury. In embodiments, the Q mitochondria may improve Ejection Fraction (EF), inhibit cardiac hypertrophy, and/or treat, prevent, ameliorate, and/or improve fibrosis after ischemia-reperfusion injury.

Exemplary autoimmune and/or inflammatory and/or fibrotic diseases and disorders include acute respiratory distress syndrome (ARDS), celiac disease, vasculitis, lupus, chronic obstructive pulmonary disease (COPD), irritable bowel disease, inflammatory bowel disease (e.g., ulcerative colitis, Crohn's disease), multiple sclerosis, atherosclerosis, arthritis, and psoriasis.

Exemplary cancers include, for example, breast cancer, ovarian cancer, cervical cancer, endometrial cancer, prostate cancer, testicular cancer, lung cancer, hepatocellular cancer, renal cancer, bladder cancer, gastric cancer, colorectal cancer, pancreatic cancer, esophageal cancer, melanoma, lymphomas, leukemias, and blastomas (e.g., neuroblastoma).

Additional diseases and disorders that may be treated by administration of the Q mitochondria provided herein include diabetes (Type I and Type II), metabolic disease (e.g., hyperglycemia, hypoglycemia, glucose intolerance, insulin resistance, hyperinsulinemia, metabolic syndrome, syndrome X, hypercholesterolemia, hypertension, hyperlipoproteinemia, hyperlipidemia, dyslipidemia, hypertriglylceridemia, kidney disease, ketoacidosis, thrombotic disorders, nephropathy, diabetic neuropathy, fatty liver, non-alcoholic fatty liver disease, and steatohepatitis), ocular disorders associated with mitochondrial dysfunction (e.g., glaucoma, diabetic retinopathy or age-related macular degeneration), hearing loss, mitochondrial toxicity associated with therapeutic agents, cardiotoxicity associated with chemotherapy or other therapeutic agents, a mitochondrial dysfunction disorder (e.g., mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome), or migraine. In embodiments, the disease or disorder is pre-eclampsia or intrauterine growth restriction (IUGR).

In embodiments, the present disclosure provides methods for treating aging and conditions associated with aging by administering the Q mitochondria provided herein to subjects in need thereof. Normal aging as well as aging-related conditions may be treated with the compositions and methods provided herein. Aging-related conditions include neurodegenerative conditions, cardiovascular conditions, hypertension, obesity, osteoporosis, cancers, and type II diabetes.

Exemplary neurodegenerative diseases and disorders include, for example, dementia, Friedrich's ataxia, amyotrophic lateral sclerosis, mitochondrial myopathy, MELAS (encephalopathy, lactic acidosis, stroke), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease.

Exemplary cardiovascular diseases include coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism.

In an aspect, the present disclosure provides methods for improving mitochondrial function in a cell, in a tissue of a subject, in an organ, in an egg cell, or in an embryo. In embodiments, the organ is heart, lung, kidney, brain, skeletal muscle, skin tissue, facial muscle, bone marrow tissue, or white adipose tissue. In embodiments, the organ is a transplanted organ. In embodiments, the cell is a transplanted cell. In embodiments, the tissue is a transplanted tissue, for example, transplanted bone marrow tissue.

In one aspect, the present disclosure provides methods for detecting mitochondrial dysfunction. In embodiments, the methods comprise detecting biomarkers of mitochondrial dysfunction. In embodiments, the present disclosure provides methods for detecting mitochondrial dysfunction in combination with a subject with the Q mitochondria provided herein if mitochondrial dysfunction is detected in the subject. In exemplary embodiments, the biomarker of mitochondrial dysfunction may be heteroplasmy, peripheral mitochondrial count, mitochondrial DNA deletion or duplication, and/or DNA methylation level. In embodiments, the biomarker may be blood levels of growth differentiation factor 15 (GDF15), apelin, humanin, and/or fibroblast growth factor 21 (FGF21).

In embodiments, the Q mitochondria provided herein are administered systemically (e.g., intranasally, intramuscularly, subcutaneously, intraarterially, intra-tracheally, via inhalation, intrapulmonary, or intravenously) or locally. In embodiments, the mitochondria are administered to the subject in a pharmaceutically acceptable carrier. In embodiments, the mitochondria are administered to the subject in combination with one or more additional agents and/or additional therapies designed to treat the disease or disorder. In embodiments, the mitochondria are syngeneic, allogeneic, or xenogenic mitochondria.

The present disclosure also provides use of Q mitochondria in the manufacture of a medicament for treating the diseases and disorders provided herein. The present disclosure also provides Q mitochondria for use in any of the methods provided herein.

The present disclosure also provides kits for use in treating the diseases and disorders provided herein. In embodiments, the kits comprise a population of Q mitochondria provided herein. In embodiments, the kits further comprise instructions for administering said Q mitochondria to a subject. In embodiments, the present disclosure also provides kits for isolating the Q mitochondria, e.g., kits for performing the iMIT isolation method. In embodiments, the kits comprise the surfactant, buffers, and instructions provided herein for isolating mitochondria from cells via the iMIT method.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### EXAMPLES

### Example 1. Detergent and Homogenization Free (DHF) Method (“iMIT”) Compared to Conventional Methods

A study was conducted to compare conventional isolation methods, which include homogenization and/or high concentration of surfactant, to a detergent and homogenization free method provided herein. The mitochondria isolated by the iMIT method are referred to herein as “Q” mitochondria. Mitochondria isolated by the homogenization and detergent methods are referred to herein as H-mitochondria or H-mito, and D-mitochondria or D-mito, respectively.

The cells used in this study were human-derived HeLa cells (RCB3680) purchased from RIKEN's cell bank. Media used for culture were passaged once or twice weekly in MEM+10% FBS. For the DHF method, the following steps were performed.

1) Cells were cultured in dishes whose diameter is 100 mm and confirmed to be 80% confluent.

2) The medium was discarded, and the cells were washed twice with 3 mL of an isolation buffer (10 mM Tris-HCl, 250 mM sucrose, 0.5 mM EGTA, pH 7.4).

3) An isolation buffer containing 3 mL of 30 μM digitonin was added and the dishes were allowed to stand still at room temperature for 3 minutes.

30 μM is about 1/10 of the critical micelle concentration (cmc) of digitonin^{(2,3)}.

4) The inside of the dish was washed twice with 3 mL of the isolation buffer.

5) 3 mL of isolation buffer was added and the dishes were allowed to stand still at 4° C. for 10 minutes.

6) The cells were detached by gentle pipetting using a micropipette.

7) Thereafter, the suspension containing the detached cells and mitochondria was transferred to a 15 mL centrifuge tube, centrifuged at 500×g, 4° C. for 10 minutes, and 2 mL of the supernatant was collected to obtain a population of the isolated mitochondria. The population of isolated mitochondria may or may not be frozen at this stage, via the following method.

8) When freezing, glycerol was added to a freezing buffer (10 mM Tris-HCl, 225 mM mannitol, 75 mM sucrose, 0.5 mM EGTA, pH 7.4) to make the glycerol concentration 10%, but not using the isolation buffer, and the frozen material of the isolated mitochondria (a population of the isolated mitochondria in frozen state or a composition containing it) was obtained by freezing with liquid nitrogen.

A second method of isolation was performed to test isolation of mitochondria using a higher concentration of surfactant (at or above the critical micelle concentration). The following steps were performed.

1) The cells were cultured in dishes whose diameter is 100 mm and confirmed to be 80% confluent.

2) The medium was discarded, and the cells were washed twice with 3 mL of the isolation buffer.

3) Digitonin dissolved in 3mL of an isolation buffer was added at a concentration of 400 μM (critical micelle concentration) and the dishes were allowed to stand still for 3 minutes at room temperature.

4) The cells were detached by gentle pipetting using a micropipette.

5) 3 mL of the suspension was transferred to a 15 mL centrifuge tube, centrifuged at 500×g at 4° C. for 10 minutes, and 2 mL of the supernatant was collected to obtain a population of the isolated mitochondria.

6) When freezing, glycerol was added to the freezing buffer to suspend it to make the concentration of glycerol 10%, and it was frozen in liquid nitrogen to obtain a frozen material of the isolated mitochondria.

A third method of isolation was performed using a conventional homogenization method and the following steps.

1) Cells were cultured in dishes whose diameter is 100 mm and confirmed to be 80% confluent.

2) The medium was discarded, and the cells were washed twice with 2 mL of an isolation buffer.

3) 3 mL of an isolation buffer was added and the cells were detached using a cell scraper.

4) The suspension of the detached cells was homogenized in a Potter-type glass Teflon (Registered trademark) homogenizer while cooling the suspension in ice. Five sets of up and down were performed for this operation.

5) The homogenate was transferred to a 15 mL centrifuge tube, centrifuged at 500×g at 4° C. for 10 minutes, and 2 mL of the supernatant was collected to obtain a population of the isolated mitochondria.

6) When freezing, glycerol was added to the freezing buffer to suspend it to make the concentration of glycerol 10%, and it was frozen in liquid nitrogen to obtain a frozen material of the isolated mitochondria.

Mitochondria isolated by each of the above-described methods were adsorbed onto glass-based dishes, and the membrane potential of individual mitochondria was observed with fluorescence microscopy. The procedures were as follows:

1) A suspension (300 μL) containing isolated mitochondria was spread on the glass surface of the glass-based dish and allowed to stand still on ice for 1 hour to immobilize the isolated mitochondria on the glass surface. Subsequently, 2 mL of 1M KOH was added to a glass-based dish (35 mm) to wash the glass surface.

2) The dishes were washed twice with 2 mL of Milli-Q water.

3) Washed with 2 mL of ethanol.

4) The dishes were washed twice with 2 mL of Milli-Q water.

5) The dishes were washed twice with 2 mL of an isolation buffer.

Using a zetasizer (Nanosize Nano-ZS, Malvern), particle size analysis, zeta potential analysis and polydispersity (PDI) analysis of the isolated mitochondria were performed according to the manufacturer's manual. Depolarization of mitochondria and mitochondrial staining with membrane-potential sensitive dyes were performed as follows.

1) The mitochondria adsorbed to the glass-based dishes were washed with 2 mL of an isolation buffer.

2) 2 mL of an isolation buffer containing 5 μM CCCP was added and the dishes were allowed to stand still at room temperature for 1 hour.

3) The buffer was replaced with 2700 μL of TMRE staining buffer containing 5 μM CCCP (10 mM Tris-HCl, 250 mM sucrose, 10 nM TMRE, 0.33 mg/mL BSA) and the dishes were allowed to stand still at room temperature in the dark for 10 minutes.

4) A total of 56 μL of malic acid and glutamic acid were added to make each of the concentrations 5 mM. Fluorescence observation was performed within 5 minutes at room temperature.

Isolated mitochondria were stained with membrane potential-sensitive dyes as follows.

1) The mitochondria adsorbed to the glass-based dishes were washed with 2 mL of an isolation buffer.

2) The buffer was replaced with 2700 μL of TMRE staining buffer (10 mM Tris-HCl, 250 mM sucrose, 10 nM TMRE, 0.33 mg/mL BSA) and the dishes were allowed to stand still at room temperature in the dark for 10 minutes.

3) A total of 56 μL of malic acid and glutamic acid were added to make each of the concentrations 5 mM. Fluorescence observation was performed within 5 minutes at room temperature.

Olympus fluorescence microscopy I×70 and a cooled CCD camera (Sensicam QE, PCO AG; Kelheim, Germany) (6.45 μm/pixel) were used for observation under the following conditions:

Objective lens: ×40, N.A. 0.9
Light source: Halogen lamp
Absorption filter: a center wavelength of 546 nm and a band pass of 10-nm
CCD camera: binning: 2×2
Exposure time: 1 second

The same field of view was observed with the same instrument as the transmitted light.

Conditions different from transmitted light were as follows:
Light source: Xenon lamp
Excitation filter: band pass filter that passes 520-550 nm
Fluorescent filters: Sharp-cut filters that passes light with 580 nm or higher

For analysis of fluorescent images, a region of 0.94 μm² was taken on individual mitochondrial transmitted light images, and the average value of fluorescence intensity within that region was obtained. The ratio of this value to the background fluorescence intensity was determined as the ratio of each mitochondrial fluorescence intensity. The fluorescence intensity ratio was calculated by rounding off to the second decimal places.

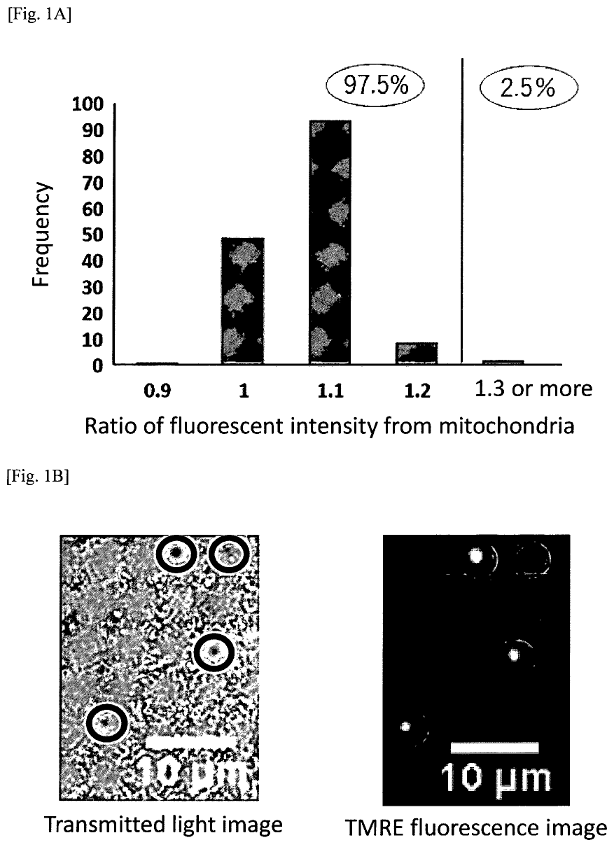

The distribution of the ratios of the fluorescence intensities was obtained from the ratios of the fluorescence intensities in depolarized mitochondria determined as described in Section 2.4.7, and the threshold value of the intensity ratio of fluorescence representing depolarized mitochondria was determined. The results are shown in FIG. 1A. As shown in FIG. 1A, more than 97% of mitochondria were present in the presence of 5 μM CCCP at a fluorescence intensity ratio of 1.2 or less. Therefore, we determined whether the mitochondria were polarized using a fluorescence intensity ratio of 1.2 as the threshold.

The proportions of polarized mitochondria were determined for the isolated mitochondria by DHF method, homogenization method, and surfactant method, respectively (n=130 to 150, respectively). The results are shown in Table 1. The polarization ratio is, for example, the percentage of black dots that appear to be 0.5-1.5 μm in diameter in the transmitted light image of the left panel of FIG. 1B, with the fluorescence intensity ratio of TMRE in the right panel of FIG. 1B being greater than the threshold value of 1.2. The mitochondria examined were before freezing.

TABLE 1
Mitochondrial polarization ratio obtained by the different methods

| methods for obtaining mitochondria | polarization ratio |
|---|---|
| DHF method (iMIT) | 90% |
| homogenization method | 38% |
| surfactant method (cmc concentration) | 38% |

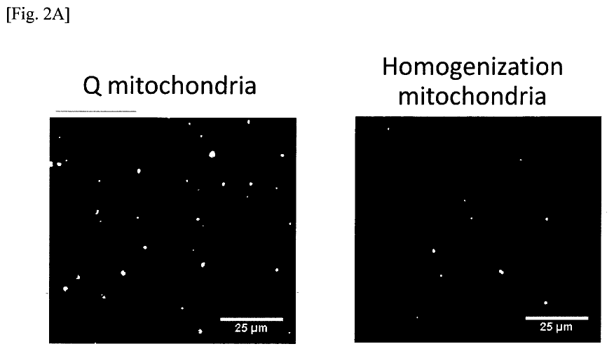

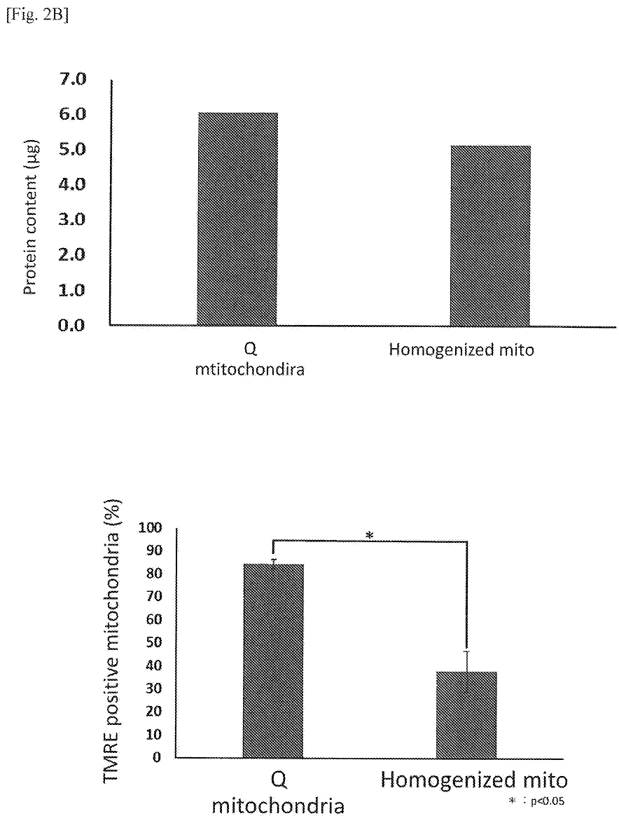

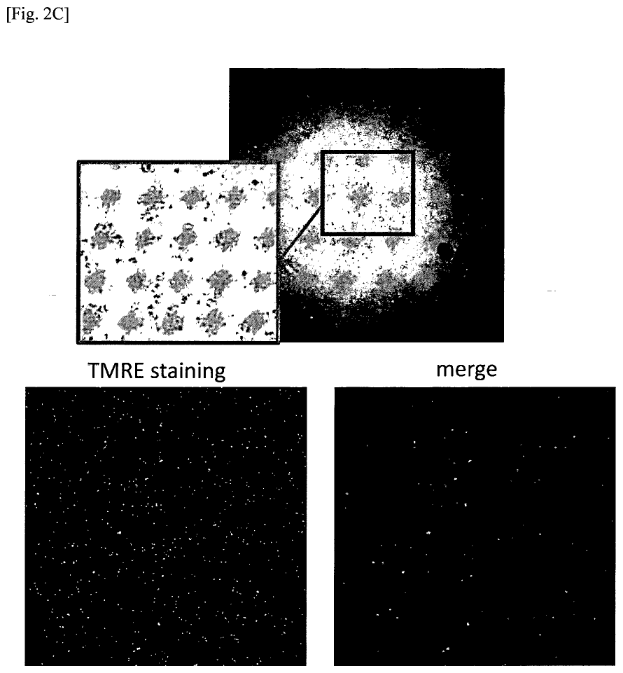

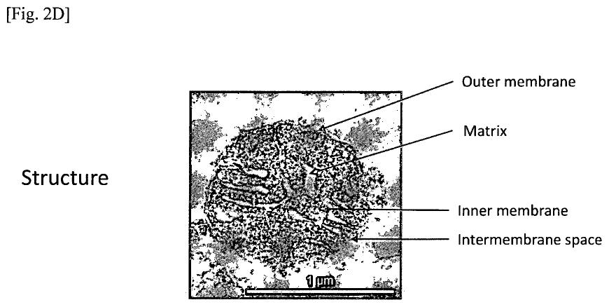

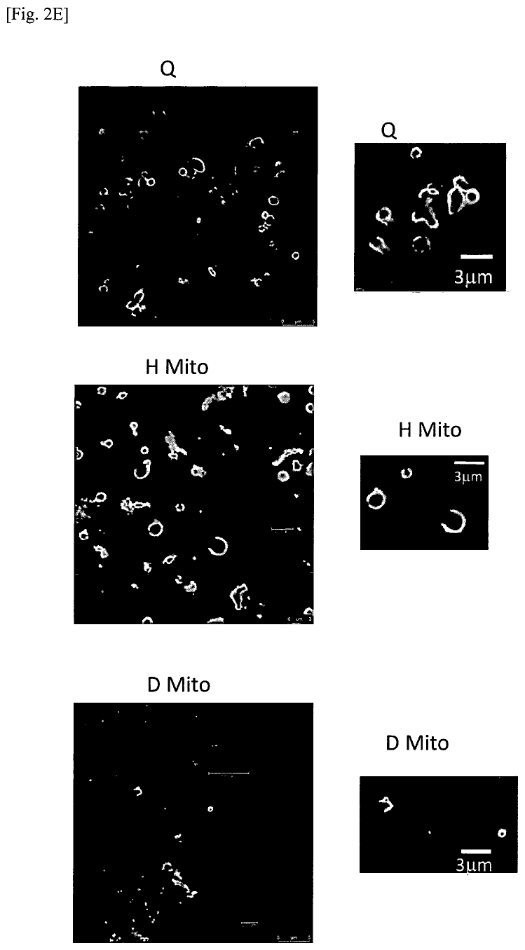

FIG. 2A shows the TMRE fluorescence of the population of Q mitochondria (iMIT method) vs. homogenization method mitochondria. Tables 1 and 2 and FIG. 2B confirm that with an equivalent number of cells at isolation and an equivalent protein content of the obtained mitochondrial fraction, the mitochondria isolated by the iMIT method exhibit a statistically significant increase the percent of TMRE positive mitochondria, compared to the conventional method (*p<0.05 in FIG. 2B, bottom panel). FIG. 2C shows that almost all mitochondria obtained via the iMIT method are TMRE+. Random selection of 10 black dots from the bright field image in comparison to the TMRE positive staining indicated that 90% of the mitochondria were TMRE positive. FIG. 2D shows that the mitochondria isolated by iMIT retain the double membrane (inner and outer membrane) and the cristae structure.

TABLE 2
Protein content and number of isolated cells obtained
via DHF method vs. homogenization method

| | Protein content of | Number of |
|---|---|---|
| | mitochondrial | cells at isolation |
| | fraction (μg) | (million) |
| DHF method mitochondria (iMIT) | 6.06 | 46 |
| Homogenized mitochondria | 5.14 | 46 |

Stimulated emission depletion (STED) microscopy was utilized to visualize Q mitochondria obtained via the iMIT method compared to mitochondria obtained via a conventional homogenization method (H mito) or conventional detergent method (D mito) (FIG. 2E). The outer membrane was stained green (immunofluorescence of Tom20) and the inner membrane was stained red using Mitotracker Red. As can be seen in FIG. 2E, Q mitochondria had intact inner and outer membranes, whereas H mito had far fewer detectable inner membranes or intact outer membranes, and D mito had even fewer detectable inner or outer membranes. Further, in H mito, it is shown that some of the inner membranes protrude from the outer membranes, suggesting that the outer membranes are physically damaged during the isolation processes. In D mito, many small debris from the outer membranes are detected, and some of the outer membranes has no inner membranes inside of the outer membranes, which suggests that the surfactant used during the isolation process would solubilize the outer membranes and inner membranes to chemically destroy the isolated mitochondria. A quantification of the ratio of mitochondria with membrane potential, the ratio of mitochondria with an intact outer membrane, the length of mitochondria along the long diameter, and the ratio of long diameter to short diameter, obtained from the study are provided below in Table 3. Protein content was also measured (mitochondria obtained from HeLa cells—1 dish 150Φ, 12 million cells). The membrane potential was measured in mitochondria isolated from HUVEC cells. Ratio of intact outer membrane, diameter, and long:short diameter were measured in mitochondria isolated from HeLa cells.

TABLE 3
Ratio of membrane potential, ratio of
intact outer membrane, and diameters

| | | Ratio | Ratio | | Long |
|---|---|---|---|---|---|
| | | main- | main- | | diameter/ |
| Method | Pro- | tained | tained | | short |
| of iso- | tein | membrane | intact outer | Long | diameter |
| lation | content | potential | membrane | diameter | ratio (r) |
| iMIT | 68 μg | 90% | 85% | 0.5-3.5 nm | 1 < r < 3.5 |
| (Q) | | | | | |
| H | 137 μg | 40% | 65% | 0.5-2 nm | 1 < r < 1.8 |
| D | 144 μg | 40% | 20% | 0.5-1.5 nm | 1 < r < 1.3 |

The results of these studies demonstrated that the iMIT method of the present disclosure is suitable for preparing mitochondria that are capable of maintaining structural integrity and exhibiting polarization. The studies also showed that mitochondria obtained by the iMIT method of the present disclosure have a higher proportion of mitochondria that can exhibit polarization (polarization ratio) than conventional mitochondrial preparation methods (homogenization and high surfactant methods). The studies also demonstrated that the mitochondria isolated via the iMIT method have a non-filamentous shape and generally are less rounded or spherical compared to the H and D mitochondria. The studies revealed that pretreatment of the cells with the surfactant with a concentration below the critical micellar concentration was sufficient for the recovery of mitochondria from the cell interior. Moreover, the studies showed that the mitochondria isolated by the iMIT method are fundamentally different, and functionally superior, compared to the mitochondria isolated by conventional methods.

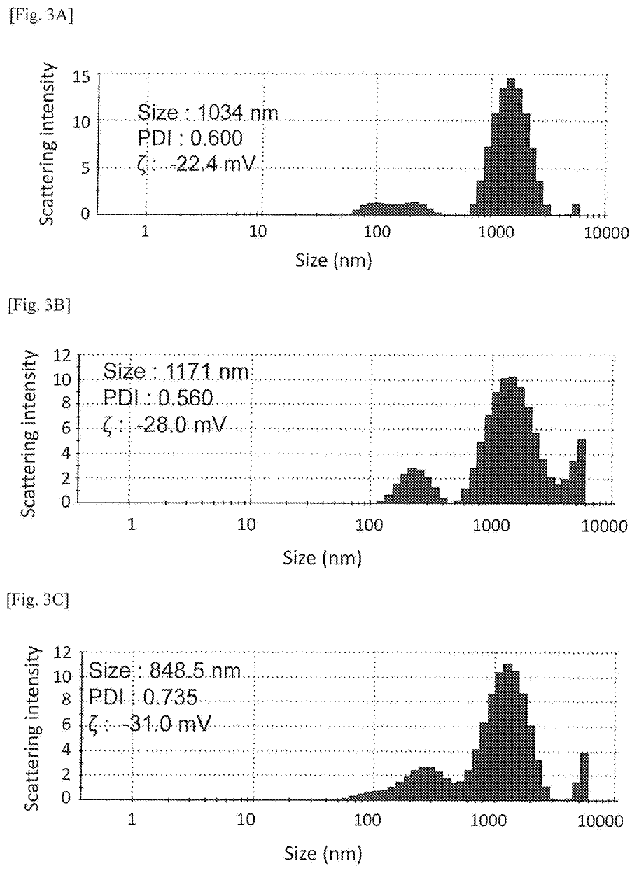

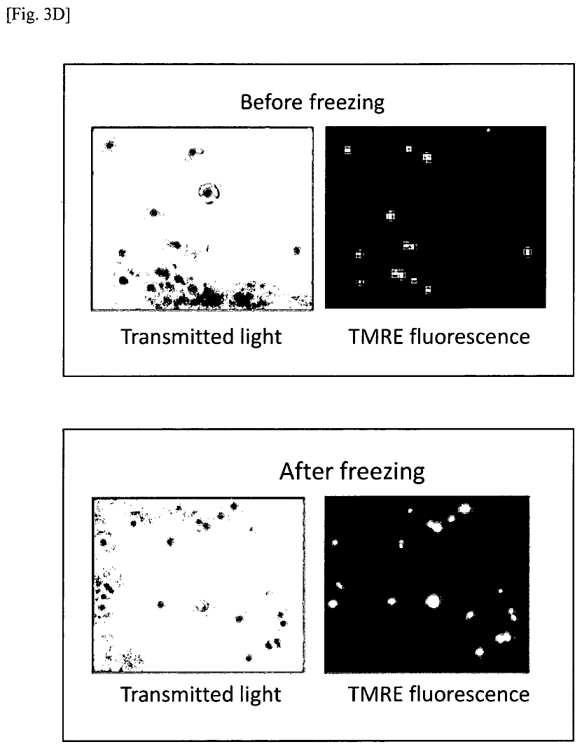

The size distribution and the zeta potential of mitochondria isolated by the iMIT method were measured. The size distribution and the zeta potential of the sample before freezing (i.e., Q mitochondria freshly isolated via the iMIT method) are shown in FIG. 3A. As shown in FIG. 3A, mitochondria isolated by the iMIT method showed monodispersity with a size (particle size) of 1034 nm. This result suggests that there is less contamination of nuclear DNA and debris derived from the cell, and that the vast majority of material recovered is mitochondria.

The size distribution and the zeta potential of the sample obtained via iMIT method after freezing and subsequent thawing are then shown in FIG. 3B. Freezing was performed by suspending the mitochondria in a freezing buffer, and then freezing the samples with liquid nitrogen. Transport of the samples to and from liquid nitrogen was performed on dry ice. Thawing was performed by holding the vial of mitochondria under running tap water while swirling the vial, such that it thawed within 3 minutes. As shown in FIG. 3B, the mitochondria isolated by the iMIT method were 1171 nm in size (particle size) even after freezing and thawing.

In addition, size distribution and zeta potential of mitochondria isolated by the iMIT method except that the centrifugation was performed at 1000×g instead of 500×g, was determined and the results are shown in FIG. 3C. After freezing and thawing the mitochondria thus isolated, and the size distribution and the zeta potential of the samples were 858.5 nm in size (particle size).

The zeta (ζ) potentials were good (between −22.4 mV and 031.0 mV) in all of the above samples.

A sample of FIG. 3A and a sample of FIG. 3B were subjected to TMRE staining and the staining superimposed on a bright-field image (merge) is shown in FIG. 3D. The results showed that the isolated mitochondria showed high polarization ratio before and after freeze-thawing.

A study was conducted to assess the use of a surfactant other than digitonin. Mitochondria were isolated from cells using saponin (concentration: approximately 40 μM, the concentration is approximately 1/15 of CMC) instead of digitonin in a similar manner as described above. The CMC of saponin is considered to be 538-646 μM (Komatsu et al., J. Oleo. Sci. 54:265-270 (2002). A good mitochondrial population was obtained by this method, with characteristics similar to those of the mitochondria obtained using digitonin.

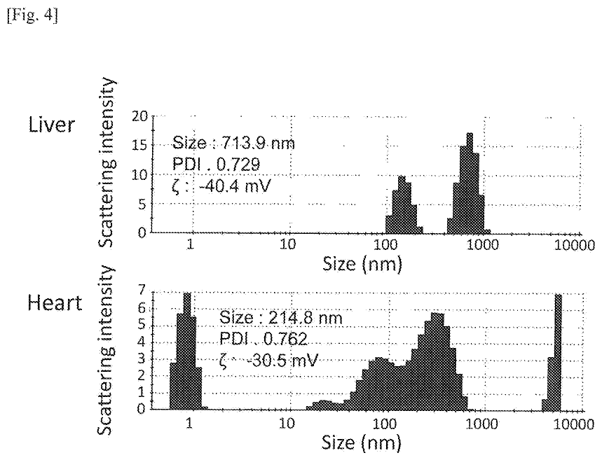

Approximately 0.1 g, 1.2 g, and 1.2 g of heart, liver, and skeletal muscle obtained from mice, respectively, were shredded and treated with collagenase (concentration: 0.2 wt %) for 30 min at 37° C. In this way, mitochondria were isolated from unicellularized cells using the iMIT method described above using digitonin below the CMC. The size, polydispersity and zeta potential of the isolated mitochondria by dynamic light scattering were measured. The results were as shown in FIG. 4. As shown in FIG. 4, mitochondria isolated from liver and heart showed good zeta potentials. Mitochondria isolated from skeletal muscle also showed good zeta potential (i.e., about −15 mV) and have a size distribution of about 100 nm to about 2,000 nm. These results indicate that mitochondria can directly be isolated from tissue samples by the iMIT method. In particular, the mitochondria obtained from a liver showed the lowest zeta potential and were considered favorable among these examples.

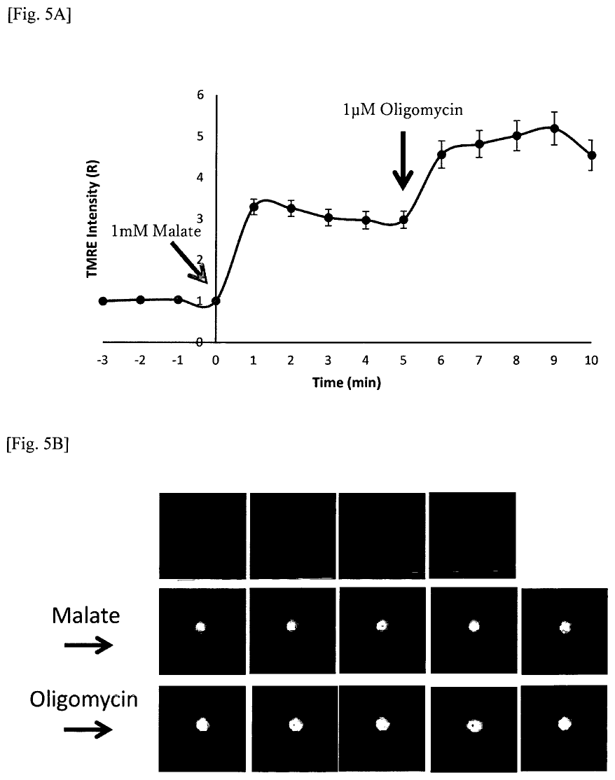

The activity of the mitochondria isolated by the iMIT method was assessed as shown in FIGS. 5A and 5B. After isolation, mitochondria were incubated for 10 minutes at room temperature in 20 nM TMRE, 1 mM KH₂PO4, 0.5 mM ADP-K, 0.33 mg/ml BSA, 0.5 M EGTA, 10 mM Tris, 110 mM sucrose, and 70 mM KCl. TMRE fluorescence was averaged over 10 mitochondria and normalized to 1 at t=0. Malate was added at 1 mM between 0 and 1 min. Oligomycin was added at 1 μM between 5 and 6 min. FIG. 5A shows the TMRE fluorescence changes in a single mitochondrion. FIG. 5B shows images of a typical time course of fluorescence images of TMRE in a single mitochondrion. The time interval between images was 1 min.

### Example 2. Co-Localization of Q Mitochondria With Endogenous Mitochondria in Recipient Cells

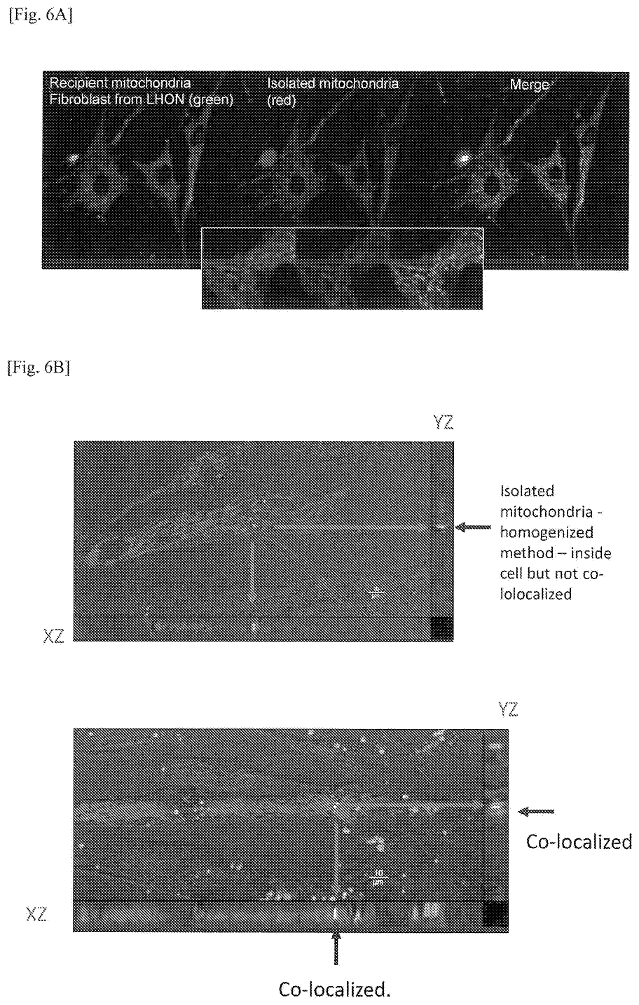

A study was conducted to determine whether mitochondria isolated via the iMIT method (Q) are capable of co-localization with mitochondria in recipient cells. Mitochondria were isolated via the iMIT method from cardio progenitor cells and stained with Mito Tracker (red). Endogenous mitochondria in recipient LHON fibroblast cells were labeled green. The exogenous, Mito tracker mitochondria were contacted with the recipient cells, and confocal microscopy images were taken. Representative images are provided in FIG. 6A and show that the isolated mitochondria move into the cells overnight and co-localized with recipient mitochondria. FIG. 6B provides a comparison of co-localization between mitochondria isolated using a conventional homogenization method and mitochondria isolated using the iMIT method provided herein. The top panel of FIG. 6B shows that a few mitochondria isolated by the conventional method appeared to move into a cell. However, far more mitochondria isolated via the iMIT method moved into a cell, and only iMIT method mitochondria co-localized with the recipient cell mitochondria to form filamentous, network-like and/or mesh-like structure (FIG. 6B, bottom panel).

### Example 3. Shape of Functional Isolated Mitochondria

A study was conducted to determine if mitochondria isolated by the iMIT method provided herein are undergoing drp1-mediated fission. In general, mitochondria in most cell types are long, filamentous, and form a network-like or mesh-like structure; any mitochondria within a cell that do not have the long filamentous and mesh-like shape, are generally non-filamentous because they are undergoing drp1-mediated fission.

In the study, a drp1 inhibitor, Mdivi1, was added to cells. Mitochondria in cells exhibit the networked and filamentous shape (FIG. 7A). Mitochondria in cells treated with the iMIT method, in contrast, were non-filamentous in shape in both the absence and presence of Mdivi1 (0 or 10 μM Mdivi1; FIG. 7B). Thus, the non-filamentous shape of the iMIT is initiated while the mitochondria are still in cells, and is not dependent on drp1 mediated fission. FIG. 7C shows the mitochondria after iMIT isolation and demonstrates that they retain the non-filamentous shape. Thus, the study indicated that the non-filamentous shape of the mitochondria isolated by the iMIT method provided herein is not dependent upon drp1 fission. That is, the Q mitochondria differ from mitochondria that are present in a cell at least in that they are non-filamentous, but are not undergoing drp1-dependent division.

### Example 4. Polarizability Under Ca2+ Conditions

A study was conducted to assess the function of Q mitochondria under high calcium conditions. Mitochondria were isolated from cells via the iMIT method, the detergent method, or the homogenization method. Each of these three populations of mitochondria were split into two subpopulations. The first subpopulation was incubated in Tris-HCl-sucrose-EGTA buffer with BSA and 10 nM TMRE as a control. The second subpopulation was incubated in DMEM containing 200 mg/mL CaCL₂ with BSA and 10 nM TMRE for 10 minutes.

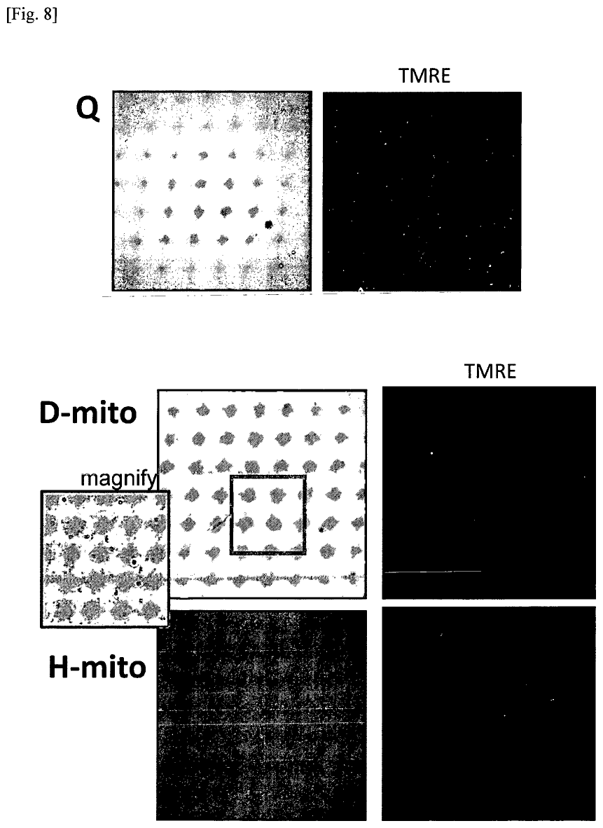

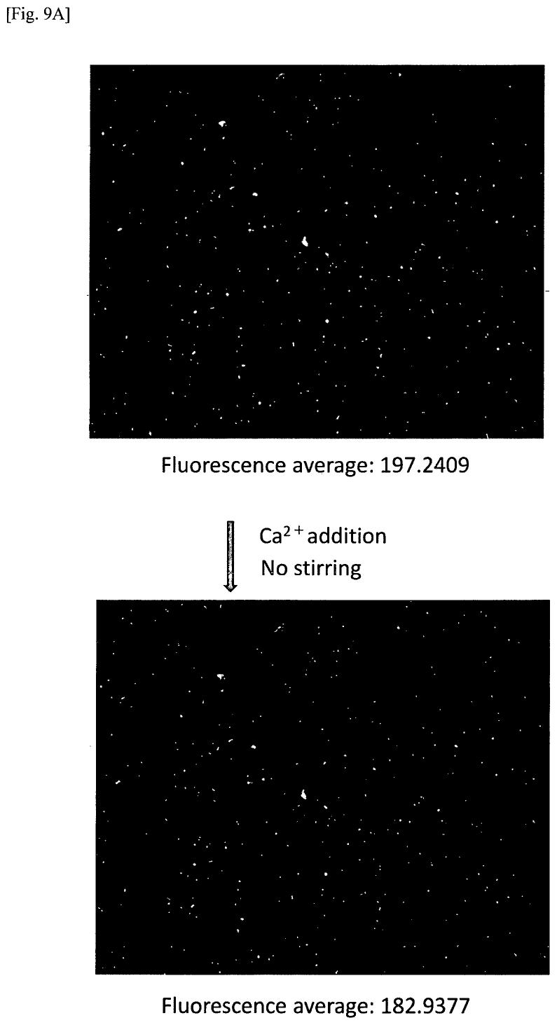

The results of the study showed that mitochondria isolated via the iMIT method (Q) showed polarizability under Ca2+ conditions, whereas the mitochondria isolated under the detergent method (D-mito) or the homogenization method (H-mito) did not show polarizability under Ca2+ conditions (FIG. 8). In the middle row of FIG. 8, magnification of the microscope image of the D-mito shows that there are many mitochondria in the sample, but the TMRE image does not show any TMRE+mitochondria. In contrast, in the top row of FIG. 8, many TMRE+ Q mitochondria are visible. Accordingly, the study showed that Q mitochondria retain the capacity to function even when exposed to a high Ca2+ environment.

Further studies were conducted using calcein fluorescence to detect holes in the mitochondrial membrane. Q mitochondria were isolated via the iMIT method from HUVEC cells and absorbed on a glass base dish. TMRE and calcein fluorescence in individual mitochondria were observed with fluorescence microscopy. Fluorescence changes upon the indicated treatments were sequentially observed in the same microscopic field.

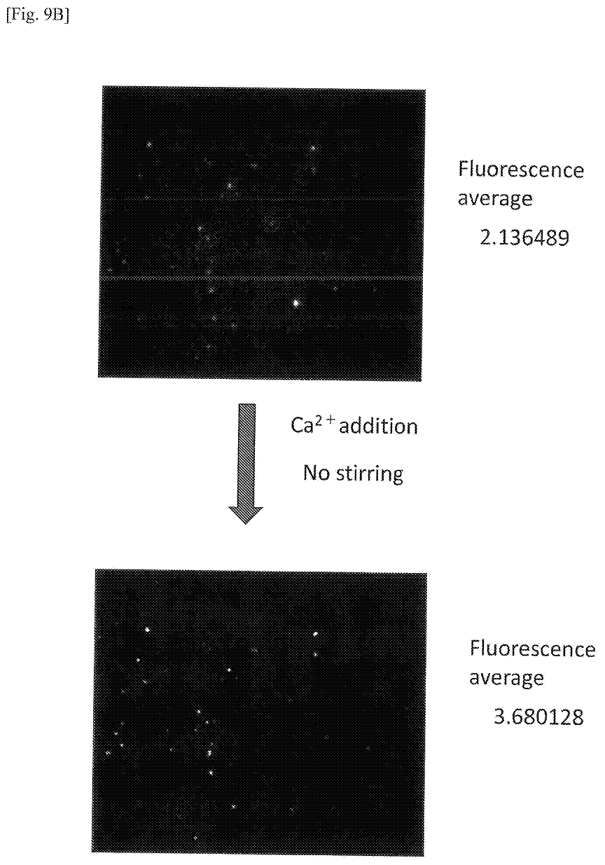

The Q mitochondria were incubated with 1 μM calcein-AM in isolation buffer containing 5 mM malate and 5 mM glutamate at room temperature for 10 minutes, and gently washed with isolation buffer. FIG. 9A, upper panel, shows calcein fluorescence in Q with 1 mL isolated buffer. (FIG. 9A). FIG. 9A, bottom panel shows the calcein fluorescence in Q after addition of 4 mL HBS (10 mM HEPES, 120 mM NaCl, 4 mM KCl, 0.5 mM MgSO4, 1 mM NaH2PO4, 4 mM NaHCO3, 25 mM glucose, 1.2 mM CaCl2, 0.1% bovine serum albumin, pH 7.4). HBS was gently added toward the edge of the dish. Thus, the addition of calcium did not change the calcein fluorescence, confirming that the Q mitochondria isolated via the iMIT method maintain membrane integrity in a Ca2+ rich environment.

The study further confirmed that the Q mitochondria in the Ca2+ rich environment maintained membrane potential as measured by TMRE fluorescence (FIG. 9B). Q were incubated with 10 nM TMRE in isolation buffer with 5 mM malate and 5 mM glutamate for 10 minutes and gently washed with isolation buffer. FIG. 9B, upper panel, shows the TMRE fluorescence in Q with 1 mL isolation buffer. Q was adsorbed at the center of glass base dish that contains 1 mL of isolation buffer. FIG. 9B, lower panel, shows the TMRE fluorescence with addition of 4 mL HBS. HBS was gently added toward the edge of the dish. TMRE fluorescence was maintained after addition of HBS.

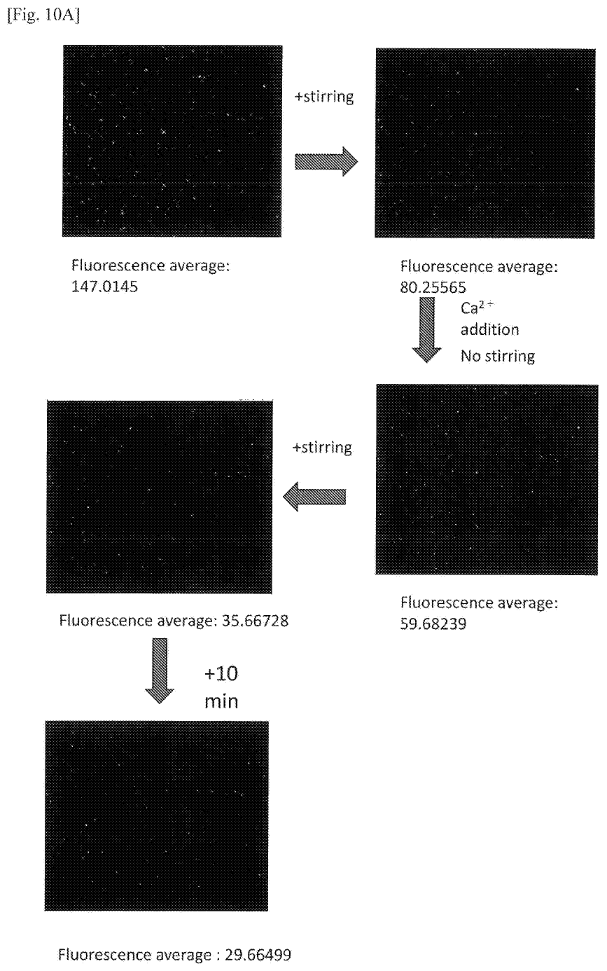

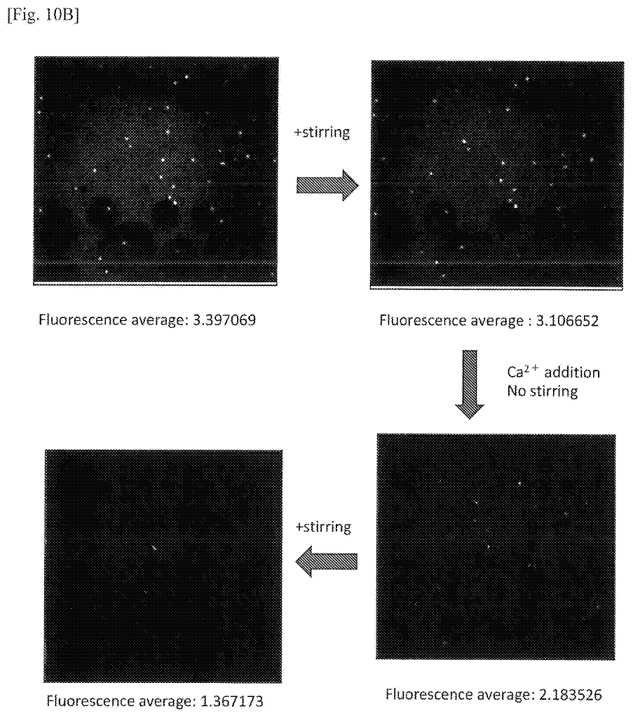

Interestingly, when the mitochondria were physically disrupted in addition to the presence of the Ca2+ environment by applying the physical stimulus of pipetting (stirring), the mitochondria lost calcein fluorescence (FIG. 10A). Q were incubated with 1 μM calcein-AM in isolation buffer with 5 mM malate and 5 mM glutamate at room temperature for 10 minutes and gently washed with isolation buffer. Agitation of the Q with pipetting (10-15 times) reduced calcein fluorescence as shown in the top right panel of FIG. 10A. Calcein fluorescence after addition of 0.96 mM Ca2+ is shown in the middle row, right panel. To add Ca2+, 4 ML of HBS with 5 mM malate and 5 mM glutamate was gently added toward the edge of the dish. Q were again agitated with 10-15 times pipetting, in the presence of Ca2+, and calcein fluorescence after addition of Ca2+ and agitation is shown in the middle row, left panel. Calcein fluorescence 10 minutes later is shown in the bottom panel of FIG. 10A. Furthermore, FIG. 10B shows that TMRE staining showed that the membrane potential (as measured by TMRE staining) was maintained despite the effect of stirring (pipetting 10-5 times) on the mitochondrial membrane; however, after exposure of the stirred mitochondria of a Ca2+ environment (0.96 mM Ca2+), the mitochondria lost membrane potential. (Q were incubated in 20 nM TMRE in isolation buffer with 5 mM malate and 5 mM glutamate at room temperature for 10 minutes. The value for fluorescence was the ratio of TMRE fluorescence in mitochondria to the background (top left panel, FIG. 10B). TMRE fluorescence after agitation with pipetting 10-15 times is shown in the top right panel of FIG. 10B. TMRE fluorescence in Q after addition of Ca2+ is shown in the bottom right panel of FIG. 10B. 4 mL of HBS with 5 mM malate and 5 mM glutamate was gently added toward the edge of the dish. TMRE fluorescence in Q in the presence of Ca2+ after agitation with 10-15 times pipetting is shown in the bottom left panel of FIG. 10B. The results of the study indicated that while the mitochondria are resistant to the Ca2+ environment, upon receiving a physical stimulus or disruption, the mitochondria may lose Ca2+ tolerance. Thus, without wishing to be bound by theory, the results suggest that physical disruption of the mitochondria (such as shaking or stirring), and/or a combination of Ca2+ rich environment with a physical disruption such as shaking or stirring may cause the mitochondria to lose membrane integrity and/or membrane potential. Without wishing to be bound by theory, the results suggested that mitochondria isolated via the iMIT method, if contacted with or stored in a Ca2+ environment, should be handled with minimal disruption, for example, prior to use in a therapeutic treatment.

### Example 5. GRP27 Content

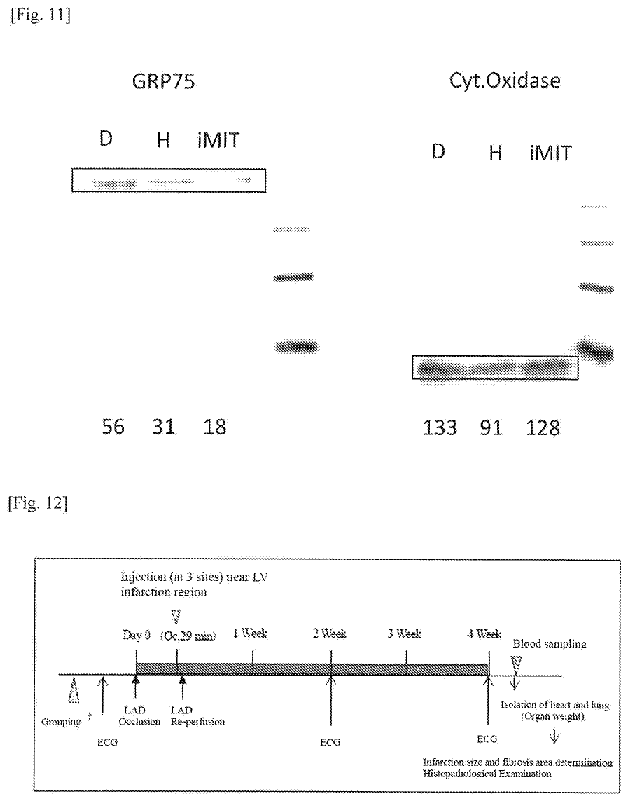

A study was conducted to compare the glucose regulated protein 75 (GRP75) content of mitochondria isolated via the iMIT method provided herein, to that of mitochondria isolated from the detergent method or homogenization method. Mitochondria were isolated by each of the three methods from HeLa cells and a western blot to detect GRP75 protein was conducted. Cytochrome oxidase was used as a protein content control. The results of the study showed that mitochondria isolated via the iMIT method provided herein had far lower GRP75 content compared to the mitochondria isolated via the detergent or the homogenization method (FIG. 11 and Table 4).

TABLE 4
GRP75 protein content comparisons

| | Detergent | Homogenization | iMIT method (Q |
|---|---|---|---|
| | method | method | mitochondria) |
| GRP75 total protein | 56 | 31 | 18 |
| content | | | |
| Cyt. Oxidase total | 133 | 91 | 128 |
| protein content | | | |
| Relative amount of | 0.42 | 0.35 | 0.14 |
| GRP75 | | | |

### Example 6. In Vivo Effect in Cardiac Infarction Model With 4 Week Follow Up

The effects on cardiac function improvement and cardiac re-modeling prevention, and safety of a local injection of Q mitochondria (isolated via the iMIT method provided herein) was evaluated using a cardiac infarction (ischemic reperfusion of coronary artery) model in rats.

The test article, “Q” mitochondrial population, was prepared by the iMIT method provided herein using HUVEC (immortalized human umbilical vein endothelial cell line, HUEhT-1). Once isolated, the mitochondria population was cryopreserved in liquid nitrogen liquid for 1-2 weeks before use. The prepared population was thawed and formulated at the study site according to internal procedures in use. Male Slc:Wister rats at an age of 11-12 weeks were used in the study, using a 30 minute left anterior descending (LAD) artery surgical occlusion to induce myocardial infarction. Animals were grouped by Stratified Random Allocation Method so that mean body weight was almost equal among the groups.

One minute before reperfusion, Q mitochondria were locally injected at 3 sites near the infarction region on the left ventricle myocardial tissue at 0.23 μg/body (low dose) or 11.5 μg/body (high dose) in 30 μL at each site, using a needle (26-30 G). PBS(−) was used as negative control. Mitochondria isolated using a conventional Mitochondria Isolation Kit (89874, Thermo Scientific) were used as a comparator at the high dose (11.5 μg/body), dosed in the same manner as the Q mitochondria. A sham group underwent the open surgery but were otherwise untreated. Ten animals each were allocated to the groups. After the dosing, body weight was measured weekly, and echocardiography was conducted at pre-dose, Week 2 and Week 4. At Week 4, blood sampling and hearts and lungs were isolated, and weights of hearts, left and right atria and ventricle and lungs. Histopathological examination was conducted using the isolated left ventricles. Additionally, size of myocardial infarction site and relative area of cardiac fibrosis were determined. General conditions of the animals were observed daily. All animals were sacrificed 4 weeks after dosing, and organ weight measurements and histopathological examinations on hearts and lungs were conducted. FIG. 12 provides a schematic of the study design.

The ischemic reperfusion (IR) model animals prepared in the present study exhibited left ventricle tissue re-modeling (enlarged LVIDs and LVIDs, and decreased LVAWd), abnormal left ventricle contraction function (decreased EF and % FS), significant increase of relative organ weights, and formation of cardiac infarction lesion and cardiac fibrosis. Additionally, histopathological examination found cardiac regenerative necrosis, inflammatory cell infiltration, interstitial edema, fibrosis, and bleedings. One animal of 10 in the control group died on day 8 after myocardial infarction model preparation (8 days after dosing). This incident was judged to be a pathological death related to myocardial infarction.

There was no significant difference in body weight in the Q groups when compared to the PBS control and conventional mitochondria control groups.

The echocardiography data is presented in Table 5. There was no significant difference in the Q groups with respect to LVIDd (diastolic left ventricular internal dimension: internal dimension at cardiac dilation), LVAWd (diastolic left ventricular anterior wall: thickness of anterior wall at cardiac dilation), or LVPWd diastolic left ventricular posterior wall: thickness of posterior wall at cardiac dilation) when compared to the PBS control or the conventional mitochondria groups.

For LVIDs (systolic left ventricular internal dimension: internal dimension at cardiac contraction), high dose Q demonstrated a significant decrease compared to PBS and conventional mitochondria control groups (p<0.05 vs PBS control, p<0.01 vs. conventional mitochondria control) (Table 5).

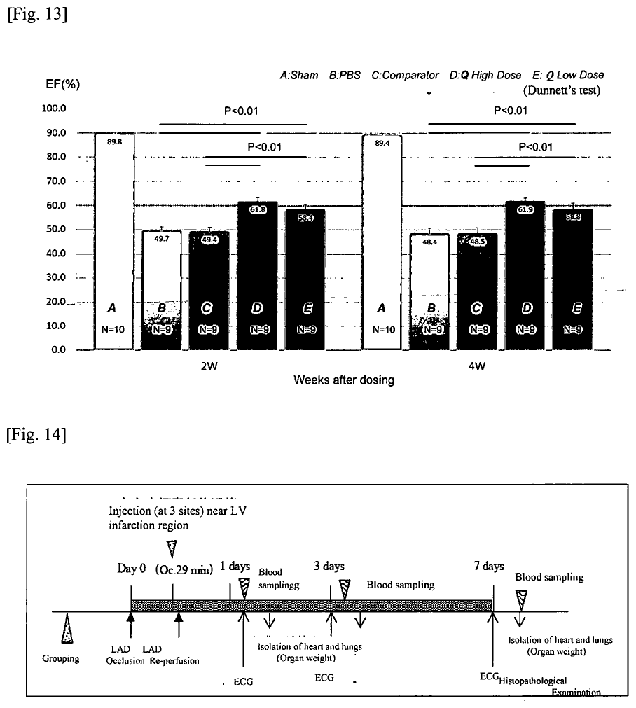

When Ejection Fraction (EF; index of total blood quantity ejected by a single cardiac contraction) was assessed, a significant increase (p<0.01) at Week 2 and Week 4 relative to PBS control and conventional mitochondria control was observed for both Q groups (Table 5). The data are also shown in FIG. 13.

For Fraction Shortening (FS; index of contraction degree in the left ventricle), significant increase (p<0.01) at Week 2 and Week 4 was also observed relative to PBS control and conventional mitochondria for both Q groups FS (Table 5).

TABLE 5
Echocardiography results

| | Dose | | | | | | | |
|---|---|---|---|---|---|---|---|---|
| | (μg/90 | | LVIDd (mm) | | | LVIDs (mm) | | |
| Group | μL) | n | Pre | 2W | 4W | Pre | 2W | 4W |
| Sham | — | 10 | 6.7 ± | 7.0 ± | 7.1 ± | 3.2 ± | 3.2 ± | 3.4 ± |
| | | | 0.1 | 0.1 | 0.1 | 0.1 | 0.1 | 0.1 |
| PBS | — | 10 | 6.5 ± | 8.6 ## ± | 9.1 ## ± | 3.1 ± | 6.8 ## ± | 7.3 ## ± |
| Control-1 | | | 0.1 | 0.2 (9) | 0.2 (9) | 0.1 | 0.2 (9) | 0.3 (9) |
| Comparator | 11.5 | 10 | 6.5 ± | 8.8 ## ± | 9.3 ## ± | 3.1 ± | 7.0 ## ± | 7.5 ## ± |
| Control-2 | | | 0.1 | 0.2 (9) | 0.2 (9) | 0.1 | 0.2 (9) | 0.2 (9) |
| QN-01 | 11.5 | 10 | 6.5 ± | 8.5 ± | 8.9 ± | 3.1 ± | * 6.1 $$ ± | * 6.5 $$ ± |
| | | | 0.1 | 0.1 (9) | 0.1 (9) | 0.1 | 0.2 (9) | 0.2 (9) |
| | 0.23 | 10 | 6.6 ± | 8.8 ± | 9.2 ± | 3.1 ± | 6.6 ± | 6.9 ± |
| | | | 0.1 | 0.1 (9) | 0.2 (9) | 0.1 | 0.2 (9) | 0.2 (9) |

| | Dose | | | | | | | |
|---|---|---|---|---|---|---|---|---|
| | (μg/90 | | LVAWd (mm) | | | LVPWd (mm) | | |
| Group | μL) | n | Pre | 2W | 4W | Pre | 2W | 4W |
| Sham | — | 10 | 1.8 ± | 1.8 ± | 1.8 ± | 1.7 ± | 1.8 ± | 1.8 ± |
| | | | 0.0 | 0.0 | 0.0 | 0.0 | 0.0 | 0.0 |
| PBS | — | 10 | 1.7 ± | 1.1 ## ± | 1.1 ## ± | 1.7 ± | 1.8 ± | 1.8 ± |
| Control-1 | | | 0.0 | 0.0 (9) | 0.0 (9) | 0.0 | 0.0 (9) | 0.0 (9) |
| Comparator | 11.5 | 10 | 1.7 ± | 1.2 ## ± | 1.1 ## ± | 1.7 ± | 1.8 ± | 1.8 ± |
| Control-2 | | | 0.0 | 0.0 (9) | 0.0 (9) | 0.0 | 0.0 (9) | 0.1 (9) |
| QN-01 | 11.5 | 10 | 1.7 ± | 1.2 ± | 1.2 ± | 1.7 ± | 1.8 ± | 1.8 ± |
| | | | 0.0 | 0.0 (9) | 0.0 (9) | 0.0 | 0.0 (9) | 0.0 (9) |
| | 0.23 | 10 | 1.8 * ± | 1.2 ± | 1.2 ± | 1.7 ± | 1.7 ± | 1.8 ± |
| | | | 0.0 | 0.0 (9) | 0.0 (9) | 0.0 | 0.0 (9) | 0.0 (9) |

| | Dose | | | | | | | |
|---|---|---|---|---|---|---|---|---|
| | (μg/90 | | EF (%) | | | % FS | | |
| Group | μL) | n | Pre | 2W | 4W | Pre | 2W | 4W |
| Sham | — | 10 | 89.4 ± | 89.8 ± | 89.4 ± | 52.9 ± | 53.5 ± | 52.8 ± |
| | | | 0.6 | 0.7 | 0.6 | 0.9 | 1.1 | 0.9 |
| PBS | — | 10 | 89.5 ± | 49.7 ## ± | 48.4 ## ± | 53.0 ± | 20.5 ## ± | 20.0 ## ± |
| Control-1 | | | 0.7 | 1.5 (9) | 2.3 (9) | 1.0 | 0.8 (9) | 1.3 (9) |
| Comparator | 11.5 | 10 | 90.0 ± | 49.4 ## ± | 48.5 ## ± | 53.8 ± | 20.4 ## ± | 20.0 ## ± |
| Control-2 | | | 0.7 | 1.6 (9) | 2.3 (9) | 1.1 | 0.9 (9) | 1.2 (9) |
| QN-01 | 11.5 | 10 | 90.1 ± | ** 61.8 $$ ± | ** 61.9 $$ ± | 53.8 ± | ** 27.6 $$ ± | ** 27.6 $$ ± |
| | | | 0.5 | 1.7 (9) | 1.3 (9) | 0.8 | 1.1 (9) | 0.8 (9) |
| | 0.23 | 10 | 89.5 ± | ** 58.4 $$ ± | ** 58.8 $$ ± | 52.9 ± | ** 25.5 $$ ± | ** 25.8 $$ ± |
| | | | 0.6 | 1.9 (9) | 2.3 (9) | 0.9 | 1.1 (9) | 1.3 (9) |

Each value represents the mean ± S.E.
Each figure in parenthesis represents the number of animals.
QN-01: Q (Test Article)
LVIDd: diastolic left ventricular internal dimension,
LVIDs: systolic left ventricular internal dimension,
LVAWd: diastolic left ventricular anterior wall,
LVPWd: diastolic left ventricular posterior wall,
EF: ejection fraction,
% FS: % fractional shortening
##: significant difference from sham at P < 0.01 (vs. Control-1 or Control-2, Student's t-test or Aspin- Welch's t-test).
* and **: significant difference between Control-1 and QN-01, at P < 0.05 and P < 0.01, respectively (Dunnett's test).
$$: significant difference between Control-2 and QN-01, at P < 0.01 (Dunnett's test).

Organ weight is presented in Table 6. There was no significant difference in overall heart weight among the groups or overall relative heart weight (organ weight of the total body weight) among the groups. However, significant suppressive effect (p<0.01) in the left atrium weight was observed in the high dose Q group compared to the conventional mitochondria control group. The relative weight of the left atrium was also significantly suppressed in the high dose Q when compared to PBS control (p<0.05) and conventional mitochondria control (p<0.01) groups. Relative weight of the right ventricle was significantly reduced in high dose Q when compared to conventional mitochondrial control group (p<0.05). Lung weight was significantly lower in high dose Q (p<0.01) and low dose Q (p<0.05) compared to conventional mitochondria control group, and relative lung weight was significantly suppressed in high dose Q compared to PBS control (p<0.05) and mitochondrial control (p<0.01) groups, and significantly suppressed in low dose Q compared to conventional mitochondrial control (p<0.05).

TABLE 6
Organ weight

| | Dose | | | | | | | | |
|---|---|---|---|---|---|---|---|---|---|
| | (μg/ | | BW | HW | RAW | LAW | RVW | LVW | LW |
| Group | 90 μg) | n | (g) | (mg) | (mg) | (mg) | (mg) | (mg) | (mg) |
| Sham | — | 10 | 335.0 ± | 773.3 ± | 35.8 ± | 25.9 ± | 138.7 ± | 573.0 ± | 1044.6 ± |
| | | | 5.4 | 12.3 | 1.2 | 1.8 | 4.3 | 8.3 | 19.8 |
| PBS | — | 9 | 331.3 ± | 882.5 ## ± | 58.6 ## ± | 40.9 ## ± | 150.0 ± | 632.9 ## ± | 1090.0 ± |
| Control-1 | | | 6.2 | 22.4 | 3.5 | 3.0 | 4.9 | 14.3 | 24.5 |
| Comparator | 11.5 | 9 | 337.8 ± | 921.5## ± | 59.1 ## ± | 44.7 ## ± | 165.0# ± | 652.8 ## ± | 1268.2 ± |
| Control-2 | | | 5.3 | 22.8 | 4.1 | 4.0 | 10.0 | 10.7 | 137.9 |
| QN-01 | 11.5 | 9 | 338.3 ± | 875.6 ± | 50.6 ± | 32.1 $$ ± | 144.9 ± | 648.0 ± | 1047.2 $$ ± |
| | | | 5.3 | 10.7 | 1.5 | 1.4 | 3.2 | 7.8 | 14.0 |
| | 0.23 | 9 | 336.7 ± | 889.8 ± | 53.0 ± | 42.3 ± | 159.6 ± | 634.9 ± | 1250.5 $ ± |
| | | | 3.5 | 21.5 | 4.0 | 3.4 | 12.6 | 11.5 | 189.0 |

| | Dose | | | | | | | |
|---|---|---|---|---|---|---|---|---|
| | (μg/ | | Relative organ weight (mg/g) | | | | | |
| Group | 90 μg) | n | HW/BW | RAW/BW | LAW/BW | RVW/BW | LVW/BW | LW/BW |
| Sham | — | 10 | 2.309 ± | 0.107 ± | 0.077 ± | 0.414 ± | 1.711 ± | 3.118 ± |
| | | | 0.021 | 0.004 | 0.005 | 0.011 | 0.014 | 0.031 |
| PBS | — | 9 | 2.664 ## ± | 0.177## ± | 0.123 ## ± | 0.452 # ± | 1.912 ## ± | 3.291 # ± |
| Control-1 | | | 0.048 | 0.009 | 0.008 | 0.011 | 0.034 | 0.053 |
| Comparator | 11.5 | 9 | 2.729 ## ± | 0.175 ## ± | 0.133## ± | 0.488 #+ | 1.933 ## ± | 3.754 ± |
| Control-2 | | | 0.060 | 0.012 | 0.012 | 0.027 | 0.02 | 0.398 |
| QN-01 | 11.5 | 9 | 2.590 ± | 0.150 ± | 0.095 *$$ ± | 0.428 $ ± | 1.917 ± | 3.097 *$$ ± |
| | | | 0.022 | 0.004 | 0.004 | 0.006 | 0.020 | 0.028 |
| | 0.23 | 9 | 2.648 ± | 0.158 ± | 0.126 ± | 0.477 ± | 1.887 ± | 3.750 $ ± |
| | | | 0.082 | 0.013 | 0.011 | 0.043 | 0.035 | 0.618 |

Each value represents the mean ± S.E.
QN-01: Q (Test Article)
BW: body weight, HW: heart weight,
RAW: right atrium weight,
LAW: left atrium weight,
RVW: right ventricular weight,
LVW: left ventricular weight,
LW: lung weight
# and ##: significant difference from sham at P < 0.05 and P < 0.01, respectively (vs. Control-1 or Control-2, Student's t-test or Aspin-Welch's t-test).
*: significant difference between Control-1 and QN-01, at P < 0.05 (Dunnett's test).
$ and $$: significant difference between Control-2 and QN-01, at P < 0.05 and P < 0.01, respectively (Dunnett's test).

There was no significant difference in myocardial infarction size, but the relative area of myocardial fibrosis was significantly smaller in high dose Q compared to conventional mitochondrial control (p<0.05) (Table 7).

TABLE 7
Fibrosis area

| | | Dose | No. of | Fibrosis area |
|---|---|---|---|---|
| Group | | (μg/90 μL) | animals | (%) |
| Sham | — | — | 10 | 0.0 ± 0.0 |
| Control-1 | PBS | — | 9 | 18.9 ± 1.1 ## |
| Control-2 | Comparator | 11.5 | 9 | 20.7 ± 1.7 ## |
| QN-01 | QN-01 | 11.5 | 9 | 16.4 ± 1.2 $ |
| | QN-01 | 0.23 | 9 | 18.6 ± 0.6 |

Each value represents the mean ± S.E.
QN-01: Q (Test Article)
##: significant difference from sham at P < 0.01 (vs. Control-1 or Control-2, Aspin-Welch's t-test).
No significant difference between Control-1 and QN-01 (Dunnett's test).
$: significant difference between Control-2 and QN-01, at P < 0.05 (Dunnett's test).

In the histopathology studies, there was no abnormality found in the specimens obtained in 10 animals in Sham Group. In all other groups, mild to moderate myocardial regenerative necrosis, mild to moderate interstitial edema, and mild to moderate fibrosis were reported.

In summary, the high dose (11.5 μg) Q group demonstrated statistically significant improvement in left ventricle tissue re-modeling (suppression of LVIDs enlargement) and left ventricle contraction function (EF and % FS) with statistically significant difference (p<0.01) when compared with negative control and comparator (conventionally isolated mitochondria) groups (FIG. 13). Significant reduction of relative organ weight (hearts and lungs) and cardiac fibrosis area was also observed in the Q groups. In histopathology examination, the degree of fibrosis and interstitial edema changes were less in the Q groups compared to that observed in negative control and comparator groups. Statistical significance (p<0.01) was also detected in Q low dose (0.23 μg) group in the improvement of left ventricle contraction function (EF, % FS) and the degree of interstitial edema, when compared with negative control and comparator groups (FIG. 13).

Taken together, based on the studies provided herein, local injection of Q mitochondria at a dose of 0.23 μg/body or 11.5 μg/body in IR model rats prevented left ventricle enlargement and improved left ventricle contractional function based on the echocardiography examination. Additionally, Q mitochondria modified increase in relative organ weight of heart and lung and cardiac fibrosis formation, according to histopathological examination outcome. The effects of Q mitochondria was higher at a dose of 11.5 μg .

### Example 7. Cardiac Infarction Model With 7 day Analyses

A second study in the cardiac infarction (ischemic reperfusion of coronary artery) rat model was performed to further assess the ability of Q mitochondria to protect against ischemic reperfusion damage. The test article, “Q,” was prepared by the iMIT method using GFP-HUVEC (green fluorescent protein labeled immortalized human umbilical vein endothelial cell line), and cryopreserved in liquid nitrogen for approximately 4 weeks prior to use in the study. In this Example, the Q mitochondria are referred to as “QN-01”.

Animals were grouped by Stratified Random Allocation Method so that mean body weight was almost equal among the groups. Two animals each were allocated for Day 1, Day 3, and Day 7 observations in Sham Group (0.23 μg labeled QN-01, with sham procedure—open surgery, but no IR preparation) and Control Group (PBS). Three animals each were allocated for the same observations in Labelled QN-01 Group (0.23 μg labelled QN-01 test group).

The animals were prepared by 30-min occlusion of left anterior descending (LAD) of rats, followed by reperfusion. As in the study described above in Example 6, QN-01 was dosed 1 min. prior to the reperfusion (after 29 min. occlusion) at a dose of 0.23 μg in the myocardial tissue, at 3 sites near the LV.

One, 3 or 7 days after the dosing, body weight was measured and echocardiography was conducted. Additionally, 1, 3 or 7 days after the dosing, blood sampling was conducted, hearts and lungs were isolated, and weights of hearts, left and right atria and ventricles and lungs were measured. All animals were subjected to organ weight measurements and histopathological examinations on hearts and lungs. The specimens obtained from the study animals were microscopically examined to find the presence of GFP, and tested by immunohistochemistry staining using anti-human mitochondrial antibody as primary antibody to investigate cellular uptake of the labeled QN-01. A schematic of the study design is provided in FIG. 14.

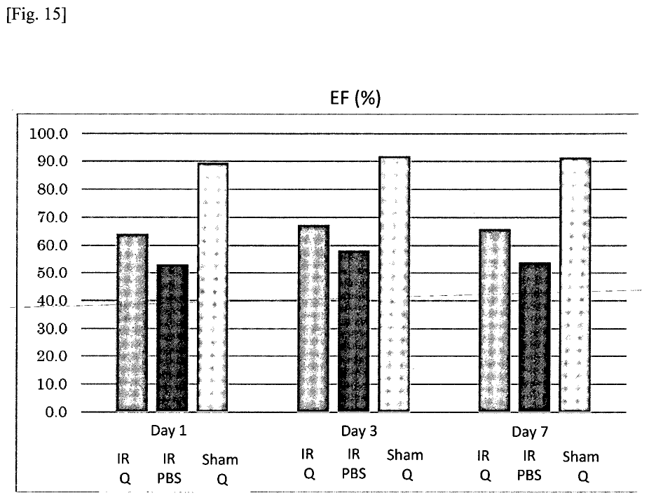

There was no death reported in the present study and all animals were monitored until the scheduled autopsy. In echocardiography, the Negative Control group exhibited decreased EF (ejection fraction), decreased % FS (fraction shortening), and enlarged LVIDs at 1, 3, and 7 days after dosing (PBS administration) and enlarged LVIDs were observed 3 and 7 days after dosing. The Q Group demonstrated superior effects in improving left ventricle contractive function (EF and % FS) when compared with Negative Control, although statistical analysis was not planned in the present study due to the limited number of animals. (FIG. 15 and Table 8). Enlargement of LVIDd and LVIDs were also suppressed compared to control (Table 9).

TABLE 8
Effect of QN-01 on echocardiographic parameters LPWD, EF, and % FS

| | | Dose | | LPWD (mm) | | | EF (%) | | | % FS | | |
|---|---|---|---|---|---|---|---|---|---|---|---|---|
| | | (μg/90 | Animal | Day | Day | Day | Day | Day | Day | Day | Day | Day |
| Group | | μL) | No. | 1 | 3 | 7 | 1 | 3 | 7 | 1 | 3 | 7 |
| Sham | QN-01 | 0.23 | Mean | 1.8 | 1.8 | 1.7 | 89.5 | 91.9 | 91.5 | 53.0 | 57.0 | 56.1 |
| | | | S.E. | 0.0 | 0.0 | — | 0.7 | 0.9 | — | 1.1 | 1.7 | — |
| Control | PBS | — | Mean | 1.8 | 1.7 | 1.6 | 53.0 | 58.1 | 53.9 | 22.4 | 25.2 | 22.8 |
| | | | S.E. | 0.0 | 0.0 | — | 2.3 | 0.4 | — | 1.2 | 0.2 | — |
| QN-01 | QN-01 | 0.23 | Mean | 1.9 | 1.9 | 1.7 | 64.0 | 67.2 | 65.8 | 29.0 | 31.1 | 30.1 |
| | | | S.E. | 0.0 | 0.0 | 0.0 | 1.6 | 1.7 | 1.1 | 1.0 | 1.1 | 0.8 |

—: no data
QN-01: Q (Test Article)
LVPWd: diastolic left ventricular posterior wall,
EF: ejection fraction,
% FS: % fractional shortening

TABLE 9
Effect of QN-01 on echocardiographic parameters LVIDd, LVIDs, AND LVAWd

| | | Dose | | LVIDd (mm) | | | LVIDs (mm) | | | LVAWd(mm) | | |
|---|---|---|---|---|---|---|---|---|---|---|---|---|
| | | (μg/90 | Animal | Day | Day | Day | Day | Day | Day | Day | Day | Day |
| Group | | μL) | No. | 1 | 3 | 7 | 1 | 3 | 7 | 1 | 3 | 7 |
| Sham | QN-01 | 0.23 | Mean | 6.5 | 6.5 | 6.8 | 3.0 | 2.8 | 3.0 | 2.0 | 2.0 | 2.0 |
| | | | S.E. | 0.1 | 0.0 | — | 0.1 | 0.1 | — | 0.0 | 0.0 | — |
| Control | PBS | — | Mean | 6.7 | 7.5 | 8.4 | 5.2 | 5.6 | 6.5 | 1.9 | 1.8 | 1.6 |
| | | | S.E. | 0.2 | 0.3 | — | 0.2 | 0.2 | — | 0.1 | 0.1 | — |
| QN-01 | QN-01 | 0.23 | Mean | 6.6 | 6.9 | 7.7 | 4.7 | 4.7 | 5.4 | 2.0 | 1.9 | 1.6 |
| | | | S.E. | 0.1 | 0.1 | 0.1 | 0.1 | 0.1 | 0.1 | 0.0 | 0.0 | 0.1 |

—: no data
QN-01: Q (Test Article)
LVIDd: diastolic left ventricle internal dimension
LIVDs: systolic left ventricle internal dimension
LVAWd: diastolic left ventricular anterior wall

In histopathology, fluorescein staining intensity was no different among dose groups including Negative Control Group, and Q was not detected in any of the specimens collected on Day 1, 3, or 7. Similarly, immunohistochemical staining test, none of the specimens tested were positive for Q detection. In the histopathological assessment, the degree of degeneration and necrosis of cardiac muscle and inflammatory cell infiltration was slightly reduced in Q Group when compared with Negative Control on Day 7 (Table 10). The plasma samples collected from all of the animals were analyzed for cytokines and chemokines using Multiplex Assay system (Luminex 17 Plex* Assay, R&D System).

TABLE 10
Histopathology findings, Day 7.

| | | Sham | | | | Control | | | | QN-01 | | | |
|---|---|---|---|---|---|---|---|---|---|---|---|---|---|
| | | (QN-01) | | | | (PBS) | | | | (QN-01) | | | |
| | | 0.23 μg/90 μL | | | | — | | | | 0.23 μg/90 μL | | | |
| | | No. of animals | | | | | | | | | | | |
| | | 2 | | | | 2 | | | | 3 | | | |
| | | Grade | | | | | | | | | | | |
| Organ/Findings | Group | − | + | ++ | +++ | − | + | ++ | +++ | − | + | ++ | +++ |
| Heart | Degeneration | 2 | | | | | | 1 | 1 | 1 | 1 | 1 | |
| | and Necrosis | | | | | | | | | | | | |
| | of cardiac | | | | | | | | | | | | |
| | muscle | | | | | | | | | | | | |
| | Inflammatory | | 2 | | | | | 2 | | | 3 | | |
| | cell infiltration | | | | | | | | | | | | |
| | Edema of | 2 | | | | 1 | 1 | | | 1 | 2 | | |
| | stroma | | | | | | | | | | | | |
| | Hemorrhage | 2 | | | | 1 | | 1 | | | 3 | | |
| | Fibrosis | | 2 | | | | | 2 | | | | 3 | |

Grade of change:
− = normal;
+ = mild;
++ = moderate;
+++ = severe

In summary, in the QN-01 Group, echocardiographic examination found that the decrease in EF and % FS 1, 3 and 7 days after dosing were suppressed compared to the PBS control group. Furthermore, enlargement of LVIDd and LVIDs were also suppressed. In histopathological examination 7 days after dosing, there was a tendency that the myocardial regenerative necrosis and inflammatory cell infiltration found in the Control Group were slightly increased relative to the QN-01 group. Accordingly, the study demonstrated that local injection of QN-01 in the myocardial infraction model rats prepared by 30 min. infarction prevented left ventricle enlargement and improved left ventricle contractional function. Additionally, QN-01 modified the degree of myocardial regenerative necrosis and inflammatory cell infiltration in heart.

### REFERENCES

1. Shibata et al., Biochem. Biophys. Res. Commun. (2015) 463, 563-8. doi: 10.1016/j.brc.2015.05.095.
2. Baker, et al. Eur. J. Transl. Myol. 2015, 25, 35-48.
3. Stetsenko and Gustov. Crystal. 2017, 7:197. https://doi.org/10.3390/cryst 7070197
4. Shin B, Cowan D B, Emani S M, Del Nido P J, McCully J D. Mitochondrial Transplantation in Myocardial Ischemia and Reperfusion Injury. Adv. Exp. Med. Biol. 2017; 982:595-619
5. Emani S M, Piekarski B L, Harrild D, Del Nido P J, McCully J D. Autologous mitochondrial transfer for dysfunction after ischemia-reperfusion injection.2)J. Thorac. Cardiovascular. Surg. 2017; 154(1):286-289
6. Bertero E, Mack C, O'Rourke B. Mitochondrial Transplantation in Humans: “Magical” cure or cause for concern? J. Clin. Invest. 2018 Dec. 3; 128(12):5191-5194
7. Segawa et al., “Quantification of cristae architecture reveals time dependent characteristics of individual mitochondria” Life Science Alliance vol. 3 no. 7, June 2020
8. Nielsen et al., The Journal of Physiology 595.9 (2017) pp. 2839-47

1. A population of isolated mitochondria, wherein:
(i) at least 80% of the mitochondria in the population have intact inner and outer membranes,
(ii) at least 80% of the mitochondria in the population are polarized as measured by a fluorescence indicator,
and/or
(iii) at least 80% of the mitochondria in the population maintain functional capabilityin an extracellular environment.

2. The population of isolated mitochondria of claim 1, wherein the functional capability in an extracellular environment of (iii) is measured by a fluorescence indicator of membrane potential.

3. The population of isolated mitochondria of claim 1, wherein the extracellular environment of (iii) comprises a total calcium concentration of about 8 to about 12 mg/dL.

4. The population of isolated mitochondria of claim 1, wherein the extracellular environment of (iii) comprises a free/active calcium concentration of about 4 to about 6 mg/dL.

5. The population of isolated mitochondria of claim 1, wherein at least 80% of the mitochondria in the population are not undergoing dynamin-related protein 1 (drp1)-dependent division.

6. The population if isolated mitochondria of claim 1, wherein the inner membranes of the mitochondria comprise densely folded cristae.

7. The population of isolated mitochondria of any one of claims 1-6, wherein at least 80% of the mitochondria in the population have a non-filamentous shape.

8. The population of isolated mitochondria of claim 7, wherein at least 85% of the mitochondria have a non-filamentous shape.

9. The population of isolated mitochondria of claim 8, wherein at least 90% of the mitochondria have a non-filamentous shape.

10. The population of isolated mitochondria of any one of claims 1-9, wherein the mitochondria exhibit decreased association with mitochondria-associated membrane (MAM) as measured by glucose regulated protein 75 (GRP75) expression.

11. The population of isolated mitochondria of claim 10, wherein the decreased association is a decrease of at least about 30% relative to the association with MAM of mitochondria in a cell or of isolated mitochondria obtained by a method comprising homogenization of cells.

12. The population of isolated mitochondria of claim 11, wherein the decreased association is a decrease of at least about 50%.

13. The population of isolated mitochondria of any one of claims 1-12, wherein
(i) at least 85% of the mitochondria in the population have intact inner and outer membranes,
(ii) at least 85% of the mitochondria in the population are polarized as measured by a fluorescence indicator,
and/or
(iii) at least 85% of the mitochondria in the population maintain functional capability in an extracellular environment.

14. The population of isolated mitochondria of any one of claims 1-12, wherein
(i) at least 90% of the mitochondria in the population have intact inner and outer membranes,
(ii) at least 90% of the mitochondria in the population are polarized as measured by a fluorescence indicator,
and/or
(iii) at least 90% of the mitochondria in the population maintain functional capability in an extracellular environment.

15. The population of isolated mitochondria of any one of claims 1-14, wherein the fluorescence indicator is selected from the group consisting of JC-1, tetramethylrhodamine methyl ester (TMRM), and tetramethylrhodamine ethyl ester (TMRE).

16. The population of isolated mitochondria of any one of claims 1-15, wherein at least 80% of the mitochondria in the population are between about 500 nm and about 3500 nm in size.

17. The population of isolated mitochondria of any one of claims 1-16, wherein the polydispersity index (PDI) of the population is about 0.2 to about 0.8,

18. The population of isolated mitochondria of any one of claims 1-16, wherein the polydispersity index (PDI) of the population is about 0.2 to about 0.3.

19. The population of isolated mitochondria of any one of claims 1-18, wherein the zeta potential of the population of mitochondria is between about −15 mV and about −40 mV.

20. The population of isolated mitochondria of any one of claims 1-19, wherein upon contact of the population of isolated mitochondria with a population of cells, the isolated mitochondria are capable co-localization with endogenous mitochondria in the cells.

21. The population of isolated mitochondria of any one of claims 1-19, wherein upon contact of the population of isolated mitochondria with a population of cells, the mitochondria are capable of fusing with endogenous mitochondria in the cells.

22. The population of isolated mitochondria of claim 20, wherein the mitochondria are capable of co-localization with the endogenous mitochondria after storage at 4° C. for at least 12 hours.

23. The population of isolated mitochondria of claim 21, wherein the mitochondria are capable of fusing with the endogenous mitochondria after storage at 4° C. for at least 12 hours.

24. The population of isolated mitochondria of any one of claims 1-23, wherein at least 70% of the isolated mitochondria in the population are polarized as measured by a fluorescence indicator, after the population undergoes one or more freeze-thaw cycle.

25. The population of isolated mitochondria of any one of claims 1-24, wherein the mitochondria are capable of co-localization with endogenous mitochondria, after the population undergoes one or more freeze-thaw cycle.

26. The population of isolated mitochondria of claim 24 or 25, wherein the population is frozen at −80° C. or colder for at least two weeks and then thawed at 20° C. or colder within about 5 minutes.

27. The population of isolated mitochondria of claim 26, wherein the population is thawed within about 1 minute.

28. The population of isolated mitochondria of claim 26 or 27, wherein the population is frozen in liquid nitrogen for the at least two weeks.

29. The population of isolated mitochondria of claim 28, wherein the population is frozen in liquid nitrogen for at least two months.

30. The population of isolated mitochondria of any one of claims 24 to 29, wherein upon contact of the thawed population of mitochondria with a population of cells, the isolated mitochondria in the population are capable of fusion with endogenous mitochondria in the cells.

31. A composition comprising the population of isolated mitochondria of any one of claims 1-30.

32. A formulation comprising the composition of claim 31 and a pharmaceutically acceptable carrier.

33. A method for isolating mitochondria from cells, the method comprising:
(i) treating cells in a first solution with a surfactant at a concentration below the critical micellar concentration for the surfactant,
(ii) removing the surfactant to form a second solution,
(iii) incubating the cells in the second solution, and
(iv) recovering mitochondria from the second solution.

34. The method of claim 33, wherein the concentration of the surfactant in the first solution is about 50% or less of the critical micelle concentration for the surfactant.

35. The method of claim 33 or 34, wherein the concentration of the surfactant in the first solution is about 10% or less of the critical micelle concentration for the surfactant.

36. The method of any one of claims 33-35, wherein the surfactant is a nonionic surfactant.

37. The method of any one of claims 33-36, wherein the surfactant is selected from the group consisting of Triton-X 100, Triton-X 114, Nonidet P-40, n-Dodecyl-D-maltoside, Tween-20, Tween-80, saponin and digitonin.

38. The method of claim 37, wherein the surfactant is saponin or digitonin, and wherein the concentration of the surfactant in the first solution is less than about 400 μM.

39. The method of claim 37, wherein the surfactant is saponin or digitonin, and wherein the concentration of the surfactant in the first solution is less than about 50 μM.

40. The method of claim 37, wherein the surfactant is saponin or digitonin, and wherein the concentration of saponin or digitonin in the first solution is about 30 μM to about 40 μM.

41. The method of any one of claims 33-40, wherein the first solution further comprises a buffer comprising one or more of a tonicity agent, osmotic modifier, or chelating agent.

42. The method of claim 41, wherein the first solution comprises a tris buffer, sucrose, and a chelator.

43. The method of any one of claims 33-42, wherein treating the cells in the first solution comprise incubating the cells in the first solution for about 2 minutes to about 30 minutes at room temperature.

44. The method of any one of claims 33-43, wherein removing the surfactant comprises decreasing the surfactant in the solution to less than 10% of the surfactant concentration in the first solution.

45. The method of any one of claims 33-44, wherein removing the surfactant comprises decreasing the surfactant in the solution to less than 1% of the surfactant concentration in the first solution.

46. The method of any one of claims 33-45, wherein removing the surfactant comprises washing the cells with a buffer.

47. The method of any one of claims 33-46, wherein incubating the second solution comprises incubating the cells in the second solution for about 5 minutes to about 30 minutes at about 4° C.

48. The method of any one of claims 33-47, wherein recovering the mitochondria from the second solution comprises collecting the supernatant to recover the isolated mitochondria.

49. The method of any one of claims 33-48, wherein recovering the mitochondria from the second solution comprises centrifuging the second solution and collecting the supernatant following centrifugation to recover the isolated mitochondria.

50. The method of any one of claims 33-49, wherein the method further comprises freezing the isolated mitochondria.

51. The method of claim 50, wherein the method comprises freezing the isolated mitochondria in a buffer comprising a cryoprotectant.

52. A population of isolated mitochondria obtained by the method according to any one of claims 33-51.

53. A method for treating a disease or disorder, the method comprising contacting cells of a subject in need thereof with a population of isolated mitochondria according to any one of claims 1-30 or a composition of claim 31 or a formulation of claim 32, wherein the disease or disorder is selected from the group consisting of diabetes (Type I and Type II), metabolic disease, ocular disorders associated with mitochondrial dysfunction, hearing loss, mitochondrial toxicity associated with therapeutic agents, cardiotoxicity associated with chemotherapy or other therapeutic agents, a mitochondrial dysfunction disorder, and migraine.

54. A method for treating a disease or disorder associated with mitochondrial dysfunction, the method comprising contacting cells of a subject in need thereof with a population of isolated mitochondria according to any one of claims 1-30 or a composition of claim 31 or a formulation of claim 32.

55. The method of claim 54, wherein the disease or disorder is selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.

56. The method of claim 54, wherein the disease or disorder is an ischemia-related disease or disorder.

57. The method of claim 56, wherein the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ.

58. The method of claim 54, wherein the disease or disorder is a genetic disorder.

59. The method of claim 54, wherein the disease or disorder is an aging disease or disorder.

60. The method of claim 54, wherein the disease or disorder is a neurodegenerative condition or cardiovascular condition.

61. The method of claim 60, wherein the neurodegenerative condition is selected from the group consisting of dementia, Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease.

62. The method of claim 60, wherein the cardiovascular condition is selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism.

63. The method of claim 54, wherein the disease or disorder is a cancer, autoimmune disease, inflammatory disease, or fibrotic disorder.

64. The method of claim 54, wherein the disease is acute respiratory distress syndrome (ARDS).

65. The method of claim 54, wherein the disease or disorder is pre-eclampsia or intrauterine growth restriction (IUGR).

66. The method of any one of claims 54-65, wherein the method comprises administering the population of isolated mitochondria or the composition to the subject via an intravenous, intra-arterial, intra-tracheal, subcutaneous, intramuscular, inhalation, or intrapulmonary route of administration.

67. An isolated mitochondrion having intact inner and outer membranes, wherein the inner membrane comprises folded cristae, wherein the mitochondrion has been isolated from a cell, wherein the mitochondrion is polarized as measured by a fluorescence indicator, and wherein the mitochondrion is capable of maintaining polarization in an extracellular environment.

68. The isolated mitochondrion of claim 67, wherein the mitochondrion has a non-filamentous shape.

69. The isolated mitochondrion of claim 67 or 68, wherein voltage dependent anion channels (VDAC) on the surface of the mitochondrion are associated with tubulin at the surface.

70. The isolated mitochondrion of claim 67, wherein the tubulin is dimeric tubulin.

71. The isolated mitochondrion of claim 70, wherein the tubulin is a heterodimer comprising α-tubulin and β-tubulin.

72. The isolated mitochondrion of any one of claims 67-71, wherein the fluorescence indicator is selected from the group consisting of JC-1, tetramethylrhodamine methyl ester (TMRM), and tetramethylrhodamine ethyl ester (TMRE).

73. The isolated mitochondrion of any one of claims 67-72, wherein the isolated mitochondrion exhibits decreased association with mitochondria-associated membrane (MAM) as measured by glucose regulated protein 75 (GRP75) expression.

74. The isolated mitochondrion of claim 73, wherein the decreased association is a decrease of at least about 30% relative to the association with MAM of a mitochondrion in a cell or of an isolated mitochondrion obtained by a method comprising homogenization of cells.

75. The isolated mitochondrion of claim 74, wherein the decreased association is a decrease of at least about 50%.

76. The isolated mitochondrion of any one of claims 67-75, wherein the membrane potential of the isolated mitochondrion is between about −30 mV and about −220 mV.

77. The isolated mitochondrion of any one of claims 67-76, wherein the isolated mitochondrion is not undergoing drp1 dependent division.

78. The isolated mitochondrion of any one of claims 67-77, wherein the isolated mitochondrion is between about 500 nm and about 3500 nm in size.

79. A composition comprising the isolated mitochondrion of any one of claims 67-78.
